SEQUENCE LISTING (12 sequences)

SEQ ID NO: 1 - DNA, 22 bp, synthetic construct
  source 1..22: mol_type = other DNA
gtgaatcatcgaatctttgaac

SEQ ID NO: 2 - DNA, 20 bp, synthetic construct
  source 1..20: mol_type = other DNA
tcctccgcttattgatatgc

SEQ ID NO: 3 - DNA, 22 bp, synthetic construct
  source 1..22: mol_type = other DNA
  modified_base 1: mod_base = OTHER; note = 6-Carboxyfluorescein modified nucleotide
  modified_base 22: mod_base = OTHER; note = Carboxytetramethylrhodamine modified nucleotide
attgcttgcggcggtaacgtcc

SEQ ID NO: 4 - DNA, 20 bp, synthetic construct
  source 1..20: mol_type = other DNA
aggcggctgtctggtctgta

SEQ ID NO: 5 - DNA, 20 bp, synthetic construct
  source 1..20: mol_type = other DNA
cctgttcgctacccatgctt

SEQ ID NO: 6 - DNA, 14 bp, synthetic construct
  source 1..14: mol_type = other DNA
  modified_base 1: mod_base = OTHER; note = 6-Carboxyfluorescein modified nucleotide
  modified_base 14: mod_base = OTHER; note = Carboxytetramethylrhodamine modified nucleotide
tgacgctgaggctc

SEQ ID NO: 7 - DNA, 25 bp, synthetic construct
  source 1..25: mol_type = other DNA
gcctacagctcagagatgctattct

SEQ ID NO: 8 - DNA, 23 bp, synthetic construct
  source 1..23: mol_type = other DNA
gccatacaccactcatgaattga

SEQ ID NO: 9 - DNA, 25 bp, synthetic construct
  source 1..25: mol_type = other DNA
  modified_base 1: mod_base = OTHER; note = 6-Carboxyfluorescein modified nucleotide
  modified_base 25: mod_base = OTHER; note = Carboxytetramethylrhodamine modified nucleotide
tggaaatgacggtcgccgttatgaa

SEQ ID NO: 10 - protein, 18 aa, synthetic construct
RRWCFRVCYRGFCYRKCR

SEQ ID NO: 11 - protein, 18 aa, synthetic construct
  SITE 1: note = Gallic acid modified residue
  SITE 4..17: note = Intrachain disulfide bond
  SITE 8..13: note = Intrachain disulfide bond
RRWCFRVCYRGFCYRKCR

SEQ ID NO: 12 - protein, 18 aa, synthetic construct
  SITE 4..17: note = Intrachain disulfide bond
  SITE 8..13: note = Intrachain disulfide bond
RRWCFRVCYRGFCYRKCR

(19) United States
(12) Patent Application Publication
(10) Pub. No.: US 2025/0090441 A1
(43) Pub. Date: Mar. 20, 2025

(54) ANTI-CARIES PEPTIDES WITH ANTIMICROBIAL AND REMINERALIZING PROPERTIES AND METHODS THEREOF

(71) Applicant: THE UNIVERSITY OF HONG KONG, HONG KONG (CN)

(72) Inventors: Yun NIU, Hong Kong (CN); Chun Hung CHU, Hong Kong (CN)

(73) Assignee: THE UNIVERSITY OF HONG KONG, HONG KONG (CN)

(21) Appl. No.: 18/805,222

(22) Filed: Aug. 14, 2024

Related U.S. Application Data
(60) Provisional application No. 63/582,649, filed on Sep. 14, 2023.
(60) Provisional application No. 63/584,538, filed on Sep. 22, 2023.

(51) Int. Cl. A61K 8/64; A61K 8/368; A61K 47/55; A61P 31/04; A61Q 11/00

(52) U.S. Cl. CPC A61K 8/64; A61K 8/368; A61K 47/552; A61P 31/04; A61Q 11/00

(57) ABSTRACT

Disclosed is an anti-caries compound, composition with antimicrobial and/or remineralizing properties and methods of using the compound and composition. The anti-caries compound is synthetic peptide Gallic-Acid-Polyphemusin-1 (GAPI) that is useful to manage dental hygiene and oral infections. Method of making a highly stable and purified form of GAPI is also disclosed. Also disclosed are the minimum inhibitory concentration and minimum bactericidal concentration against common oral pathogens.

### RELATED APPLICATIONS

The present application claims priority to U.S. provisional application Ser. No. 63/582,649 filed Sep. 14, 2023, and U.S. provisional patent application No. 63/584,538, filed Sep. 22, 2023 which are incorporated by reference in their entireties.

### SEQUENCE LISTING

The instant application contains a Sequence Listing which has been submitted electronically in XML file format and is hereby incorporated by reference in its entirety. Said XML copy, created on Oct. 15, 2024, is named 10030_012093-US1_SL.xml and is 16,942 bytes in size.

### 1. FIELD

Disclosed is a compound, composition with antimicrobial and/or remineralizing properties and methods of using the compound and composition.

### 2. BACKGROUND

Antimicrobial peptides are antimicrobial molecules produced by multicellular or unicellular organisms. Most antimicrobial peptides are oligopeptides consisting of 12-50 amino acids. The typical secondary structures of antimicrobial peptides are α-helical, β-sheet, loop and extended structures (Zhang et al. 2023b). Antimicrobial peptides mainly form amphipathic conformations in active states with abundant cationic or hydrophobic residues. This cationic amphipathic structure allows antimicrobial peptides to selectively target the lipidic bacterial membrane and the negatively charged microbial surface. Antimicrobial peptides can damage the microbial cytoplasmic membrane in the form of membrane pore or membrane disintegration. The interaction of antimicrobial peptides with the cytoplasmic membrane results in increased membrane permeability and finally leads to membrane lysis and cell content release (Mai et al. 2017). Some hypothetical models, such as ‘barrel-stave,’ ‘carpet,’ ‘toroidal-pore’ or ‘detergent-like’ model, have been proposed to illustrate the mechanism of membrane disruption of antimicrobial peptides (Luo and Song 2021). In addition to membrane destruction, antimicrobial peptides interact with intracellular substances after penetration into the cytoplasm. They can inhibit activities of intracellular enzymes and interfere with synthesis of nucleic acids, protein and cell wall (Mai et al. 2017). Antimicrobial peptides specifically target invading pathogens and produce selective toxicity without damaging the host cell. Moreover, antimicrobial peptides rarely induce resistance mutations because they usually attack multiple hydrophobic and polyanionic bacterial targets (Andersson et al. 2016). Thus, antimicrobial peptides are potential alternatives to traditional antimicrobial agents for use as therapeutic agents (Zhang et al. 2023a). Apart from killing pathogens, antimicrobial peptides also have various functions and may play modulatory roles in innate and acquired immunity.

Antimicrobial peptides have antimicrobial properties against oral pathogenic bacteria. They can also enhance tissue healing and protect teeth and oral mucosa (Tao et al. 2005). The role of antimicrobial peptides is critical for controlling multiple oral diseases, including dental caries (Niu et al. 2021a). Cariogenic microbes are essential for caries development. The definition of cariogenic microorganisms includes the following factors: (1) the bacteria have strong bond affinity to the tooth surface; (2) the bacteria can synthesize extracellular and intracellular polysaccharides; (3) the bacteria are acidogenic, transporting and metabolizing various carbohydrates; and (4) the bacteria can tolerate acid environments (Chen et al. 2020). Streptococcus, Lactobacillus, and Actinomyces species are three common cariogenic microorganisms' taxa.

Oral diseases are a global public health problem affecting over 3.5 billion people Worldwide (Peres et al. 2019). They can start in early childhood and progress throughout adolescence, adulthood, and old age (Kassebaum et al. 2017). Oral diseases have substantial negative effects on individuals, communities, and the wider society. The global economic burden of dental diseases amounts to more than USD 442 billion yearly (Listl et al. 2015). The most prevalent oral diseases are dental caries and periodontal disease, which, when left untreated, can progress to tooth loss (Tonetti et al. 2017).

Dental caries is one of the most common chronic diseases worldwide. It results from dissolution of tooth mineral by the acidic by-products of bacterial fermentation (Pitts et al. 2017). Controlling cariogenic biofilm and maintaining tooth minerals are both essential for managing dental caries. Traditional antibiotics have limitations for caries management. The excessive use of antibiotics could lead to bacterial resistance and alter the oral and intestinal microbiota. In addition, antibiotics have no remineralizing properties. Hence, it is important to develop an effective anti-caries agent with both antimicrobial and remineralizing properties. Given the diverse species and functional modifications of antimicrobial peptides, it is an objective to develop an anti-caries peptide with both antimicrobial and remineralizing properties.

### 3. SUMMARY

Provided herein is a novel synthetic anti-caries peptide Gallic-Acid-Polyphemusin-I (GAPI) peptide by fusing Gallic acid to Polyphemusin-I. In one embodiment, provided is a method of controlling cariogenic biofilm and maintaining tooth minerals for managing dental caries. Disclosed is a dual-function peptide with antimicrobial and remineralizing properties. GAPI is biocompatible to human gingival fibroblasts. In certain embodiments, it inhibits the growth of common cariogenic species including Streptococcus mutans, Lacticaseibacillus casei and Candida albicans in planktic and biofilm phase. It also remineralize dentine and enamel caries in laboratory conditions. Also provided is a composition comprising the GAPI of the present disclosure. In certain embodiments, the composition is a pharmaceutical composition.

In one embodiment, provided is a method of administering GAPI to a subject by managing caries. In one embodiment, the subject is high caries risk population.

Provided herein is a Gallic-Acid-polyphemusin-1 (“GAPI”) molecule having a structure (SEQ ID NO: 11):

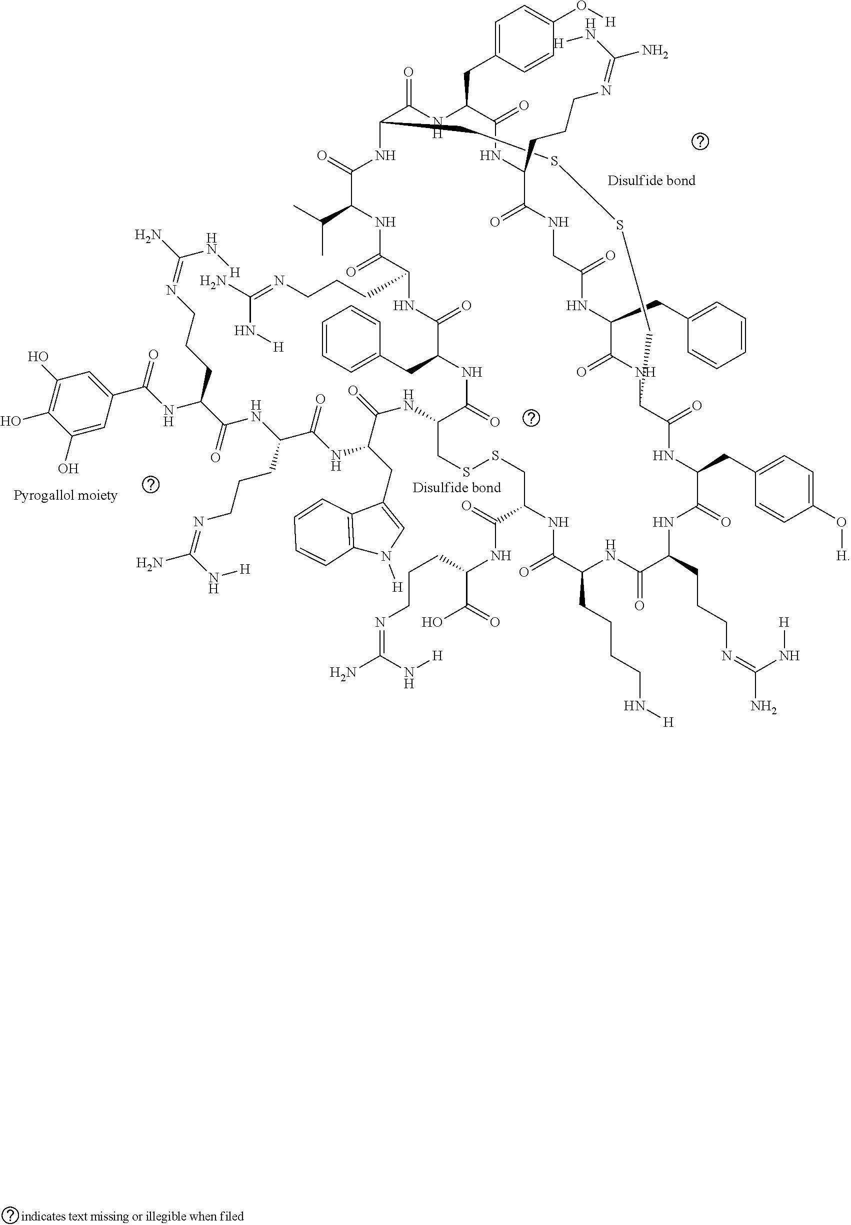

In certain embodiments, the molecule has a purity percentage from 90.0% to 99.9%.

In certain embodiments, the molecule has a molecular weight ranges from about 2600 kD to 2624 kD with a proportion of the R-sheet of from 40.0% to 56.0%.

In certain embodiments, the molecule comprises antimicrobial and remineralizing properties.

In certain embodiments, the molecule is stable for at least 60 minutes in saliva and maintain more than 90% in saliva after 60 minutes.

Provided herein is a method of making Gallic-Acid-polyphemusin-1 (“GAPI”) molecule comprising reacting gallic acid with Polyphemusin-I and forming disulfide bonds at C4-C17 and C8-C13.

In certain embodiments, the GAPI has a purity percentage of 90.0% to 99.9%.

In certain embodiments, the GAPI has a molecular weight ranges from about 2600 kD to 2624 kD with a proportion of the R-sheet of about 40.0% to 56.0%.

In certain embodiments, the molecule comprises antimicrobial and remineralizing properties.

In certain embodiments, the molecule is stable for at least 60 minutes in saliva and maintain more than 90% in saliva after 60 minutes.

Provided herein is a method of inhibiting cariogenic biofilm and remineralizing dentine in a subject, said method comprises administration of an effective amount of the molecule described in the present disclosure.

In certain embodiments, the remineralizes dentine occurs in early enamel caries.

In certain embodiments, the method further prevents collagen degradation of dentine.

In certain embodiments, the cariogenic biofilm is produced by cariogenic species selected from the group consisting of Streptococcus mutans, Lacticaseibacillus casei and Candida albicans.

In certain embodiments, the remineralizing dentine is measured by a chemical pH cycling model comprising Micro-CT scans.

In certain embodiments, minimum inhibitory concentration (“MIC”) against Streptococcus mutans (ATCC 35668), Streptococcus mutans (UA159), Lacticaseibacillus casei and Candida albicans is 80 μM, 40 μM, 40 μM and 20 μM, respectively and minimum bactericidal concentration (“MBC”) is 160 μM, 80 μM, 160 μM and 40 μM, respectively.

In certain embodiments, MICs against S. mutans and S. sobrinus are 80 μM, wherein the MBCs are 160 μM and 320 μM, respectively.

In certain embodiments, MICs against L. acidophilus and L. rhamnosus are 40 μM and 20 μM, and the MBCs are 80 μM and 160 μM, respectively.

In certain embodiments, MICs and MBCs for A. naeslundii and E. faecalis are 160 μM and 640 M, respectively.

In certain embodiments, MICs for P. gingivalis and A. actinomycetemcomitans are 320 μM and 160 μM, respectively and the MBCs for P. gingivalis and A. actinomycetemcomitans are 640 μM and 320 μM, respectively.

In certain embodiments, MICs and MBCs against cariogenic species range from 20 to 320 μM and 80 to 640 μM, respectively.

In certain embodiments, the molecule is stable for at least 60 min in saliva and maintain more than 90% in saliva after 60 minutes.

In certain embodiments, the cariogenic biofilm lost its three-dimensional structure upon administration of the molecule.

### 4. BRIEF DESCRIPTION OF THE FIGURES

The patent or application file contain at least one drawing executed in color.

FIG. 1. Synthetic Polyphemusin-I (SEQ ID NO: 10)

FIG. 2. β-turn Polyphemusin-I (SEQ ID NO:12)

FIG. 3. Gallic acid

FIG. 4. Gallic-Acid-Polyphemusin-I (GAPI) (SEQ ID NO: 11)

FIG. 5. High-performance liquid chromatography for Gallic-Acid-Polyphemusin-I (GAPI) peptide and Polyphemusin-I peptide. The area percentage of the major peak indicated the purity of the testing peptide. The purity percentages of GAPI and polyphemusin-I were 96.74% and 96.91%, respectively.

FIG. 6. Mass spectrometry analysis for Gallic-Acid-Polyphemusin-I (GAPI) peptide and Polyphemusin-I peptide. The molecular weight was 2608.05 kD for GAPI and 2455.30 kD for polyphemusin-I. The molecular weight of the synthesised peptide was 152.92 kD more than that of polyphemusin-I. It is equal to the molecular weight of gallic acid minus a water molecule due to polymerisation.

FIG. 7. Circular dichroism spectroscopy spectrum for Gallic-Acid-Polyphemusin-I (GAPI) peptide and Polyphemusin-I peptide. The secondary structure was modelled using CDPro software. The secondary structure analysis of GAPI and polyphemusin-I showed that the proportions of the β-sheet were 48.0% and 73.1%, respectively.

FIG. 8. Concentration of GAPI and optical density values of human gingival fibroblasts. The biocompatibility of GAPI was evaluated by cytotoxicity assay using Cell Counting Kit-8 Assay. Human gingival fibroblast cells were cultured and treated with GAPI. The cells cultured with the only medium were negative controls. The optical density values indicating the number of viable cells was measured at 450 nm after 24 h of incubation. There was no significant difference in optical density values between the 80, 160, 320 and 640 μM GAPI-treated HGF-1 cells and the negative control cell. It revealed that GAPI showed low cytotoxicity to HGF-1 cells.

FIGS. 9A-9F. represent the morphology of various cariogenic bacteria that were treated with or without GAPI. (FIG. 9A) S. mutans was severely damaged after being treated with GAPI. The S. mutans cells lost their normal morphology, with effects including abnormal cell curvatures and irregular cell shapes. The cell wall separated from the cell membrane. In addition, the cells' cytoplasmic membranes were entirely disrupted, resulting in transparent cytoplasmic zones and the leakage of cytoplasmic contents. (FIG. 9B) For GAPI-treated S. sobrinus, the morphology changes were similar to GAPI-treated. S. mutans: the abnormal curvature of cell membranes and irregular cell shapes, clear cytoplasmic zones, the disruption of the cytoplasmic membrane, and the leakage of cytoplasmic contents. (FIG. 9C) For L. acidophilus, (FIG. 9D) L. rhamnosus, and (FIG. 9E) A. naeslundii, the typical changes after treatment with GAPI included the abnormal curvature of cell membranes, irregular cell shapes, and cytoplasmic clear zones. (FIG. 9F) For E. faecalis, compared with untreated bacteria, higher magnification images showed that the bacteria in the GAPI group had abnormal morphological characteristics, including the disruption of the cytoplasmic membrane and the leakage of cytoplasmic contents.

FIGS. 10A-10B represent the morphology of various periodontal-associated bacteria with or without GAPI treatment. For (FIG. 10A) P. gingivalis and (FIG. 10B) A. actinomycetemcomitans, after being treated with GAPI, the abnormal curvature of cell membranes, irregular cell shapes, and intra-bacterial vacuolization can be identified. In addition, membrane disruption and the leakage of intracellular components were observed.

FIG. 11. Transmission electron micrographs of the bacteria and the fungi before and after Gallic-Acid-Polyphemusin-I (GAPI) peptide treatment. The morphology of the bacteria and the fungi treated with GAPI was analyzed using transmission electron microscopy. Streptococcus mutans treated with GAPI exhibited abnormal cell membrane curvature and disrupted cytoplasmic membrane with leakage of cytoplasmic contents. Lacticaseibacillus casei treated with GAPI exhibited irregular cell shape and prominent cytoplasmic clear zones. Candida albicans treated with GAPI exhibited abnormal cell membrane curvature and irregular cell shape.

( [symbol] Abnormal cell morphology; [symbol] Cytoplasmic clear zone; [symbol] Disrupted membrane/cell wall; [symbol] Cytoplasmic content leakage)

[symbol]

FIGS. 12A-12C. Three-species biofilm with or without Gallic-Acid-Polyphemusin-I (GAPI) peptide treatment. (FIG. 12A) Scanning electron micrographs of the biofilm with or without GAPI treatment. GAPI treatment inhibited biofilm formation and altered cell morphology. (FIG. 12B) Confocal laser scanning micrographs of the biofilm with or without GAPI peptide treatment. GAPI reduced the microorganisms. Candida albicans (green) could be distinguished from bacterial cells (pink). (FIG. 12C) Log 10 cell counts of the bacteria/fungi in a three-species biofilm with or without GAPI peptide (n=6 per group).

FIGS. 13A-13C. Enamel treated with 160 μM Gallic-Acid-Polyphemusin-I (GAPI) peptide, 160 μM Gallic Acid (GA), 160 μM Polyphemusin-I (PI) peptide or deionized water (Water). (FIG. 13A) Scanning electron micrographs of the enamel in four groups. The micrographs showed a smooth enamel and homogenous prism pattern in the GAPI Group and the GA Group compared to PI Group and Water Group. In the PI Group and Water Group, damaged enamel prisms were found in the prism and inter-prism regions. (FIG. 13B) Microcomputed tomographs of the enamel in four groups. The figures showed an undamaged enamel surface in the GAPI Group and the GA Group, but a demineralized surface in the PI Group and the Water Group. The lesion depths and mineral loss of GAPI-treated enamel were less than the Water Group. (FIG. 13C) X-ray diffraction patterns of the enamel in four groups. The reflections of GAPI Group and GA Group were sharper compared with the other groups. The reflection peak of the four groups at full width and half maximum were 0.237°, 0.342°, 0.231° and 0.328°. These results indicated that the crystallinity of GAPI-treated enamel was higher than that treated with water.

FIGS. 14A-14C. (FIG. 14A) CLSM images (×100) and (FIG. 14B) SEM images (×2000 and (FIG. 14C) ×20,000) of the S. mutans biofilm. The CLSM images showed dead (red)/live (green) fluorescence of the S. mutans biofilm. The dead-live ratio for the GAPI-treated group was significantly higher than that for the control group (p<0.001). The SEM images showed the structure of the S. mutans biofilm. The bacterial coverage in the GAPI-treated group was lower than that in the control group. In addition, GAPI treatment disrupted the bacterial structure, including loss of normal bacterial cell morphology and damage to bacterial cell membranes.

FIGS. 15A-15B Micro-CT images of dentine lesion treated with (FIG. 15A) GAPI and (FIG. 15B) water. The lesion depth for the GAPI-treated group was significantly lower than that in the control group (p<0.001).

FIG. 16 Fourier transform infrared spectra of the dentine caries lesion. The HPO₄²⁻ band wavelength from 900 to 1200 cm⁻¹ and the amide I band wavelength from 1585 to 1720 cm⁻¹. The HPO₄²⁻-to-amide I ratio for the GAPI-treated group was significantly higher than that of the control group (p=0.014).

FIGS. 17A-17B. SEM images (×7000 and ×15,000) of Dentinetreated with (FIG. 17A) 160 μM GAPI peptide or (FIG. 17B) deionized water (Water). Scanning electron micrographs showed a smooth dentine with the ball-form mineral nodes indicating remineralization and slight exposure of collagen fibres in the GAPI Group. In the Water Group, the dentine surface was rough, and the exposure of collagen fibers was considerably apparent.

FIGS. 18A-18B show typical dentine's cross-sectional morphology. The cross-sectional images exhibited that the dentinal tubular and inter-tubular regions were relatively full of mineral nodes, with slight dentin collagen fiber exposure in the GAPI group. SEM images (×7000 and ×15,000) of cross-section dentine treated with (FIG. 18A) GAPI and (FIG. 18B) water. The dentinal tubular and inter-tubular regions were relatively full of mineral nodes in the GAPI-treated group. In contrast, the dentin collagen fiber was exposed in the control group.

FIG. 19 displays the typical characteristic XRD spectra of dentin. In the GAPI group, hydroxyapatite diffraction peaks (211, 300, and 310) were identified at 31.8°. 32.9°, and 39.8°. These peaks suggested the existence of hydroxyapatite crystals. In contrast, the spectra in the control group showed no prominent diffraction peaks. X-ray diffraction patterns of the dentine caries lesion. The hydroxyapatite diffraction peaks including 211, 300, and 310 were identified in the GAPI-treated group, suggesting the existence of hydroxyapatite crystals. In contrast, the control group showed no prominent diffraction peaks.

FIG. 20 Study protocol. Before the solutions were transformed, each block was washed in an ultrasonic bath. The blocks in the GAPI group received a 5 min. topical application of GAPI using micro brushes (ROCODENT, Foshan, China), and the blocks in the control group received deionized water.

### 4.1 DEFINITIONS

The terms “subject,” “individual,” and “patient” may be used interchangeably and refer to humans, as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

The term “peptide” as used herein refers to a polymer of amino acid residues typically ranging in length from 2 to about 18 residues. In certain embodiments, the peptide ranges in length from about 2, 3, 4, 5, 7, 9, 10, 11, 12, 13, 14, 15, 16, 17, 18 residues. In certain embodiments, the peptide ranges in length from about 8, 9, 10, 11, or 12 residues to about 15, 16, 17, or 18 residues. In certain embodiments, the amino acid residues comprising the peptide are “L-form” amino acid residues, however, it is recognized that in various embodiments, “D” amino acids can be incorporated into the peptide. Peptides also include amino acid polymers in which one or more amino acid residues is an artificial chemical analogue of a corresponding naturally occurring amino acid, as well as to naturally occurring amino acid polymers. In addition, the term applies to amino acids joined by a peptide linkage or by other, “modified linkages” (e.g., where the peptide bond is replaced by an α-ester, a β-ester, a thioamide, sulfonamide or phosphoramide, carbamate or carbonate, hydroxylate, and the like (see, e.g., Spatola, (1983) Chem. Biochem. Amino Acids and Proteins 7: 267-357), where the amide is replaced with a saturated amine (see, e.g., Skiles et al., U U.S. Pat. No. 4,496,542, which is incorporated herein by reference, and Kaltenbronn et al., (1990) Pp. 969-970 in Proc. 11th American Peptide Symposium, ESCOM Science Publishers, The Netherlands, and the like)).

The term “residue”” as used herein refers to natural, synthetic, or modified amino acids. Various amino acid analogues include, but are not limited to 2-aminoadipic acid, 3-aminoadipic acid, beta-alanine (beta-aminopropionic acid), 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4 diaminobutyric acid, desmosine, 2,2′-diaminopimelic acid, 2,3-diaminopropionic acid, N-ethylglycine, N-ethylasparagine, hydroxylysine, allo-hydroxylysine, 3-hydroxyproline, 4-hydroxyproline, isodesmosine, allo-isoleucine, N-methylglycine, sarcosine, N-methylisoleucine, 6-N-methyllysine, N-methylvaline, norvaline, norleucine, ornithine, and the like. These modified amino acids are illustrative and not intended to be limiting.

The terms “conventional” and “natural” as applied to peptides herein refer to peptides, constructed only from the naturally-occurring amino acids: Ala, Cys, Asp, Glu, Phe, Gly, His, Ile, Lys, Leu, Met, Asn, Pro, Gln, Arg, Ser, Thr, Val, Trp, and Tyr. A compound of the disclosure corresponds to a natural peptide if it elicits a biological activity (e.g., antimicrobial activity) related to the biological activity and/or specificity of the naturally occurring peptide. The elicited activity may be the same as, greater than or less than that of the natural peptide. In general, such a peptoid will have an essentially corresponding monomer sequence, where a natural amino acid is replaced by an N-substituted glycine derivative, if the N-substituted glycine derivative resembles the original amino acid in hydrophilicity, hydrophobicity, polarity, etc.

Where an amino acid sequence is provided herein, L-, D-, or beta amino acid versions of the sequence are also contemplated as well as retro, inversion, and retro-inversion isoforms. In addition, conservative substitutions (e.g., in the binding peptide, and/or antimicrobial peptide, and/or linker peptide) are contemplated. Non-protein backbones, such as PEG, alkane, ethylene bridged, ester backbones, and other backbones are also contemplated. Also fragments ranging in length from about 5, 6, 7, 8, 9, 10, 11, 12, 13, 14, 15, 16, 17, 18 amino acids up to the full length minus one amino acid of the peptide are contemplated where the fragment retains at least 50%, preferably at least 60% 70% or 80%, more preferably at least 90%, 95%, 98%, 99%, or at least 100% of the activity (e.g., binding specificity and/or avidity, antimicrobial activity, etc.) of the full length peptide are contemplated.

An “antimicrobial peptide” or “AMP” may comprise two or more AMPs joined together. The AMPs can be joined directly or through a linker. They can be chemically conjugated or, where joined directly together or through a peptide linker can comprise a fusion protein.

In certain embodiments, conservative substitutions of the amino acids comprising any of the sequences described herein are contemplated. In various embodiments one, two, three, four, or five different residues are substituted. The term “conservative substitution” is used to reflect amino acid substitutions that do not substantially alter the activity (e.g., antimicrobial activity and/or specificity) of the molecule. Typically, conservative amino acid substitutions involve substitution one amino acid for another amino acid with similar chemical properties (e.g. charge or hydrophobicity). Certain conservative substitutions include “analog substitutions” where a standard amino acid is replaced by a non-standard (e.g., rare, synthetic, etc) amino acid differing minimally from the parental residue. Amino acid analogs are considered to be derived synthetically from the standard amino acids without sufficient change to the structure of the parent, are isomers, or are metabolite precursors.

In certain embodiments, antimicrobial peptides comprising at least 80-85%, at least 85-90% or 90-95%, and at least 95-98% sequence identity with any of the sequences described herein are also contemplated. The terms “identical” or percent “identity,” refer to two or more sequences that are the same or have a specified percentage of amino acid residues that are the same, when compared and aligned for maximum correspondence, as measured using one of the following sequence comparison algorithms or by visual inspection. With respect to the peptides of this disclosure sequence identity is determined over the full length of the peptide. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman (1981) Adv. Appl. Math. 2: 482, by the homology alignment algorithm of Needleman & Wunsch (1970) J Mol. Biol. 48: 443, by the search for similarity method of Pearson & Lipman (1988) Proc. Natl. Acad. Sci., USA, 85: 2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

The term “specificity” when used with respect to the antimicrobial activity of a peptide indicates that the peptide preferentially inhibits growth and/or proliferation and/or kills a particular microbial species as compared to other related and/or unrelated microbes. In certain embodiments, the preferential inhibition or killing is 10-20% greater (e.g., LD.sub.50 is 10% lower), 20-30%, 30-40%, 40-50%, or 50-60%, 2-4 fold, 5-10 fold, or 10-20 fold greater for the target species.

The term antimicrobial peptide (AMP) as described herein, comprises the peptide or peptides, variants, analogues, or derivatives thereof that possess substantially the same or greater antimicrobial activity and/or specificity as the referenced peptide. In certain embodiments, substantially the same or greater antimicrobial activity indicates at least 80%, preferably at least 90%, and more preferably at least 95% of the antimicrobial activity of the referenced peptide(s) against a particular bacterial species.

As used herein, the term “about” refers to a measurable value such as an amount, a time duration, and the like, and encompasses variations of ±20%, ±10%, ±5%, ±1%, ±0.5%, or ±0.1% from the specified value.

The term “effective amount” or “therapeutically effective amount” as used herein refers to the amount of a pharmaceutical composition comprising one or more peptides as disclosed herein or a mutant, variant, analog or derivative thereof, to decrease the level of Streptococcus mutans and prevent tooth decay, and relates to a sufficient amount of pharmacological composition to provide the desired effect. A therapeutically or prophylactically significant reduction in S. mutans is, for example, about 10-20%, about 20-30%, about 30-40%, about 40-50%, about 50-60%, about 60-70%, about 70-80%, about 80-90%, about 90-95%, at least 2 log 10, at least 3 log 10, at least 4 log 10, or at least 5 log 10 or more in CFU/mL as compared to a control or non-treated subject or the state of the subject prior to administering the oligopeptides described herein. Measured or measurable parameters can include clinically detectable markers of disease, for example, within the process of tooth decay. It will be understood, however, that the total daily usage of the compositions and formulations as disclosed herein will be decided by the attending physician or dentist within the scope of sound professional judgment.

“Treating” or “treatment” of a condition as used herein may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

### 5. DETAILED DESCRIPTION

Described is a synthesized an antimicrobial peptide and a composition comprising the antimicrobial peptide. In one embodiment, the antimicrobial peptide is an anti-caries peptide Gallic-Acid-Polyphemusin-I (“GAPI”) peptide by fusing Gallic acid to Polyphemusin-I. Polyphemusin I (PI) is an antimicrobial peptide derived from horseshoe crabs. It can kill bacteria through binding to and by crossing cell membranes, thus rupturing the bacterial membrane (Zhang et al. 2019). Gallic acid is abundant in fruits and vegetables, and it can accelerate the regeneration of hydroxyapatites due to its pyrogallol group. In addition, gallic acid shows antimicrobial activities (Niu et al. 2022).

The GAPI was biocompatible to human gingival fibroblasts. Its minimum inhibitory concentration against Streptococcus mutans, Lacticaseibacillus casei and Candida albicans were 40 μM, 40 μM and 80 μM, respectively. Transmission electron microscopy showed that GAPI can damage the morphology of bacterial and fungal cells. The architecture of a consortium biofilm consisting of Streptococcus mutans, Lacticaseibacillus casei and Candida albicans after GAPI treatment was evaluated using scanning electron microscopy (SEM) and confocal laser scanning microscopy. The results showed that the cells lost their typical morphology and the biofilm lost its three-dimensional structure. Growth kinetics of biofilm was examined using propidium monoazide-quantitative polymerase chain reaction. GAPI inhibited the growth of the three microorganisms. The GAPI-treated enamel after acid-challenge was evaluated using SEM and energy-dispersive X-ray spectroscopy (EDS) for morphological examination and elemental analysis. SEM showed that the GAPI-treated enamel had smoother surface with homogenous prism pattern than the enamel treated with deionized water (control). EDS demonstrated the calcium-to-phosphorus molar ratio of enamel treated with GAPI was higher than that of the control. Microcomputed tomography found that lesion depths and mineral loss of GAPI-treated enamel were less than the control. Using X-ray diffraction, crystallinity of GAPI-treated enamel was higher than the control. In conclusion, this study developed a biocompatible, remineralizing and antimicrobial peptide GAPI which could be used as an anti-caries agent.

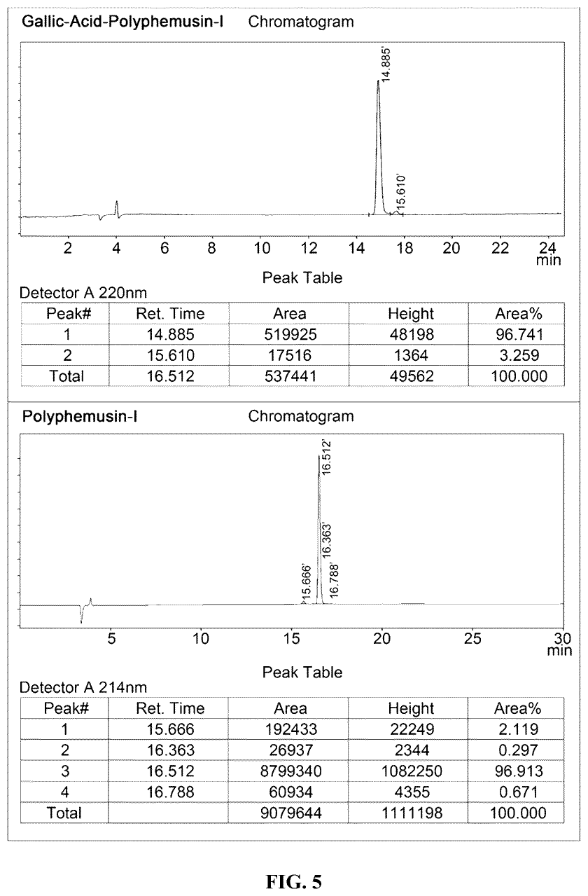

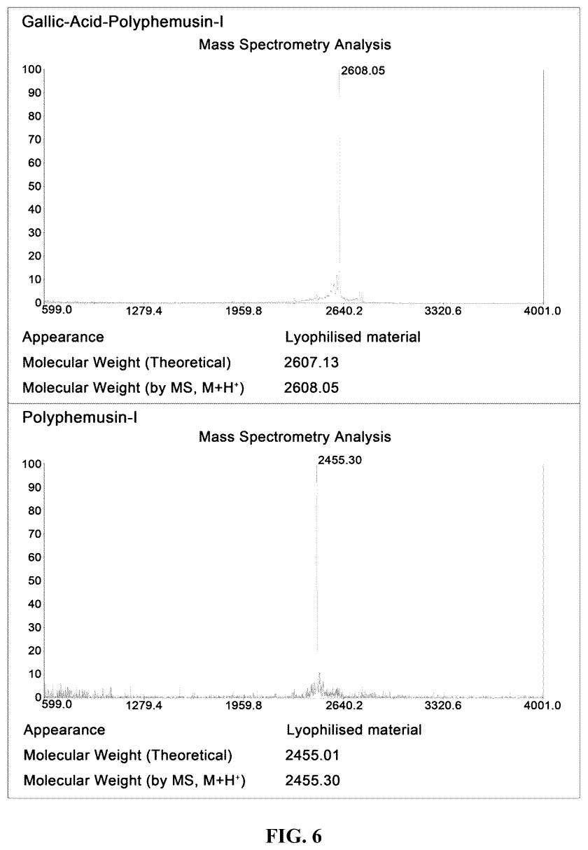

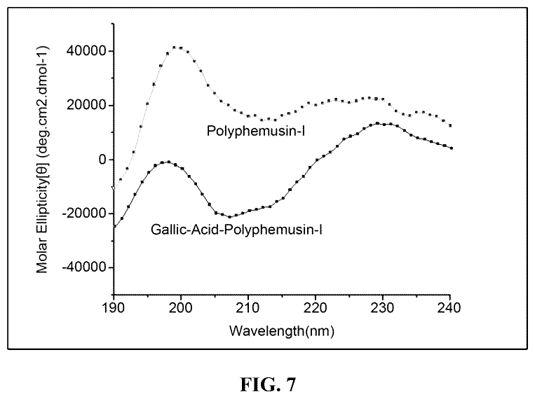

The purity percentages of GAPI and polyphemusin-I were 96.74% and 96.91%, respectively (FIG. 5). The molecular weight was 2608.05 kD for GAPI and 2455.30 kD for polyphemusin-I (FIG. 6). The secondary structure analysis of GAPI and polyphemusin-I showed that the proportions of the β-sheet were 48.0% and 73.1%, respectively (FIG. 7). The remaining percentages of GAPI after incubation in human saliva for 15 min, 30 min, 45 min and 60 min were 98.9%, 98.6%, 96.8% and 94.4%, respectively. There was no significant difference in optical density values between the 80, 160, 320 and 640 μM GAPI-treated HGF-1 cells and the negative control cell (FIG. 8). It revealed that GAPI showed low cytotoxicity to HGF-1 cells.

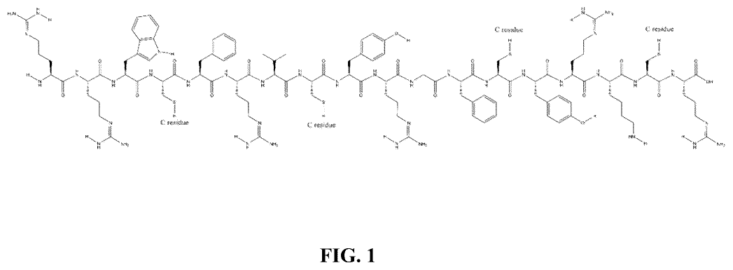

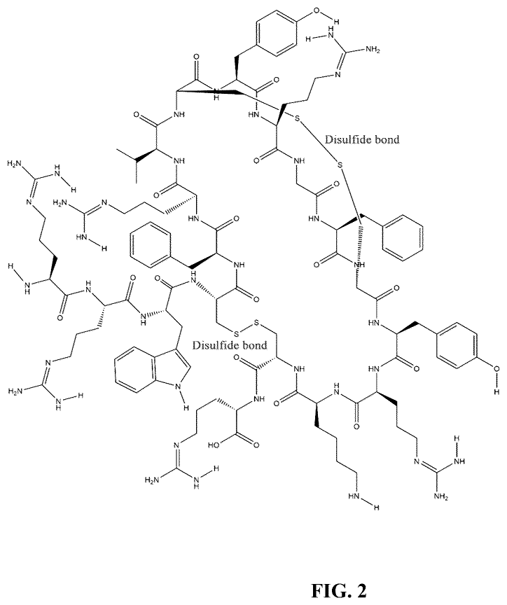

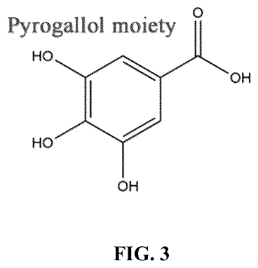

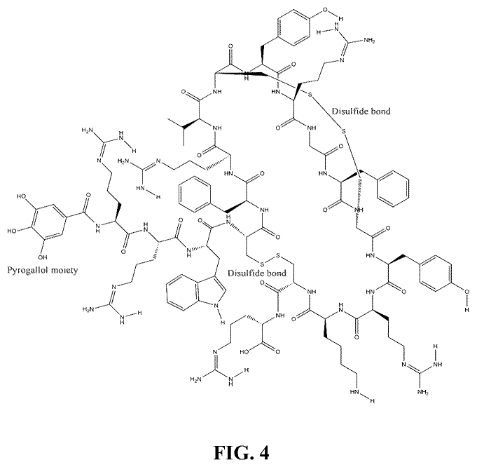

Polyphemusin-I (FIG. 1) is an antimicrobial peptide derived from horseshoe crabs having antimicrobial properties against Streptococcus mutans. Antimicrobial peptide such as DPS-PI, comprising synthetic Polyphemusin-I such as in FIG. 1. However, this type of antimicrobial peptide doesn't show demineralizing properties. In addition, the structure of synthetic Polyphemusin-I is unstable due to the tendency of C residues to form disulfide bonds. The present inventors fixed the position of disulfide bonds to form 3-turn Polyphemusin-I (FIG. 2). The pyrogallol moiety of gallic acid (FIG. 3) strongly binds to calcium ions and accelerates regeneration of hydroxyapatite. The inventors are the first to graft “Gallic acid” and “Polyphemusin-I”. The inventors encountered a purity issue when attempted to graft these compounds, with a purity of only 81.60%. To solve this problem, the present inventors fixed the position of disulfide bonds at C4-C17 and C8-C13 during the synthesis of Polyphemusin I. This stabilized the structure of the synthetic Polyphemusin I, resulting in a purity of 96.74% of GAPI after grafting with gallic acid.

### 5.1 Antimicrobial Properties

MIC and MBC/MFC values of GAPI and polyphemusin-I against tested bacteria and fungi are shown in Table 1 below. The morphology of microorganisms treated with GAPI displayed severe defects in TEM micrographs (FIG. 5). Streptococcus mutans (UA159) treated with GAPI exhibited abnormal cell membrane curvature and disrupted cytoplasmic membrane with leakage of cytoplasmic contents. Lacticaseibacillus casei treated with GAPI mainly exhibited irregular cell shape and prominent cytoplasmic clear zones. As for GAPI-treated Candida albicans, the primary defect was abnormal cell membrane curvature and irregular cell shape.

TABLE 1
Minimum inhibitory concentration (MIC)
and minimum bactericidal/fungicidal concentration
(MBC/MFC) of the two peptides

| | Gallic-Acid- Polyphemusin- | | Polyphemusin- | |
|---|---|---|---|---|
| | I (GAPI) peptide | | I (PI) peptide | |
| Bacteria/fungus | MIC | MBC/MFC | MIC | MBC/MFC |
| Streptococcus mutans | 40 | 80 | 20 | 80 |
| Lacticaseibacillus | 40 | 160 | 20 | 160 |
| casei | | | | |
| Candida albicans | 20 | 40 | 0.3 | 0.6 |

The MIC and MBC of GAPI against Streptococcus mutans, Streptococcus sobrinus, Lactobacillus acidophilus, Lactobacillus rhamnosus, Actinomyces naeslundii, Enterococcus faecalis, Porphyromonas gingivalis, and Actinobacillus actinomycetemcomitans are summarized in Table 1B.

TABLE 1B
The minimum inhibitory concentration (MIC) and minimum
bactericidal concentration (MBC) of GAPI against common
American Type Culture Collection (ATCC) oral pathogens.

| Bacteria | ATCC | MIC (μM) | MBC (μM) |
|---|---|---|---|
| Actinobacillus | 29523 | 160 | 320 |
| actinomycetemcomitans | | | |
| Actinomyces naeslundii | 12104 | 160 | 640 |
| Enterococcus faecalis | 29212 | 160 | 640 |
| Lactobacillus acidophilus | 9224 | 40 | 80 |
| Lactobacillus rhamnosus | 10863 | 20 | 160 |
| Porphyromonas gingivalis | 33277 | 320 | 640 |
| Streptococcus mutans | 35668 | 80 | 160 |
| Streptococcus sobrinus | 33478 | 80 | 320 |

The MICs of GAPI against S. mutans and S. sobrinus were 80 μM, whereas the MBCs for these two bacteria were 160 μM and 320 μM, respectively. For L. acidophilus and L. rhamnosus, the MICs were 40 μM and 20 μM, and the MBCs were 80 μM and 160 μM, respectively. The MICs and MBCs for A. naeslundii and E. faecalis were 160 μM and 640 μM, respectively. The MICs for P. gingivalis and A. actinomycetemcomitans were 320 μM and 160 μM, respectively. The MBCs for P. gingivalis and A. actinomycetemcomitans were 640 μM and 320 μM, respectively. The results indicated that GAPI showed strong antimicrobial activity against cariogenic bacteria.

According to the results of the present study, GAPI exhibited significant antibacterial efficiency. The MICs and MBCs against eight bacteria were shown to range from 20 to 320 μM and 80 to 640 μM, respectively, which are better than the other peptides from existing studies (MICs and MBCs ranged from 160 to 320 μM and 640 to 1280 μM, respectively) (Niu et al. 2021d).

### 5.2. Morphology of the Microorganisms

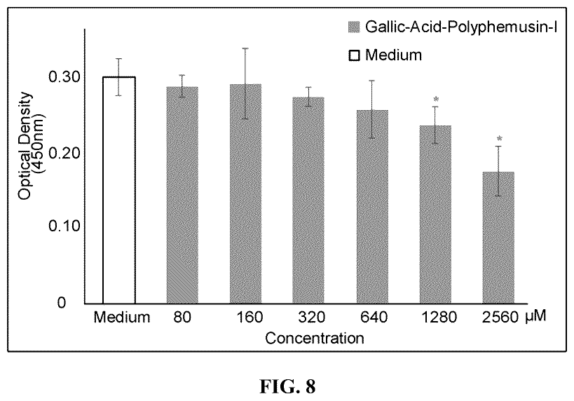

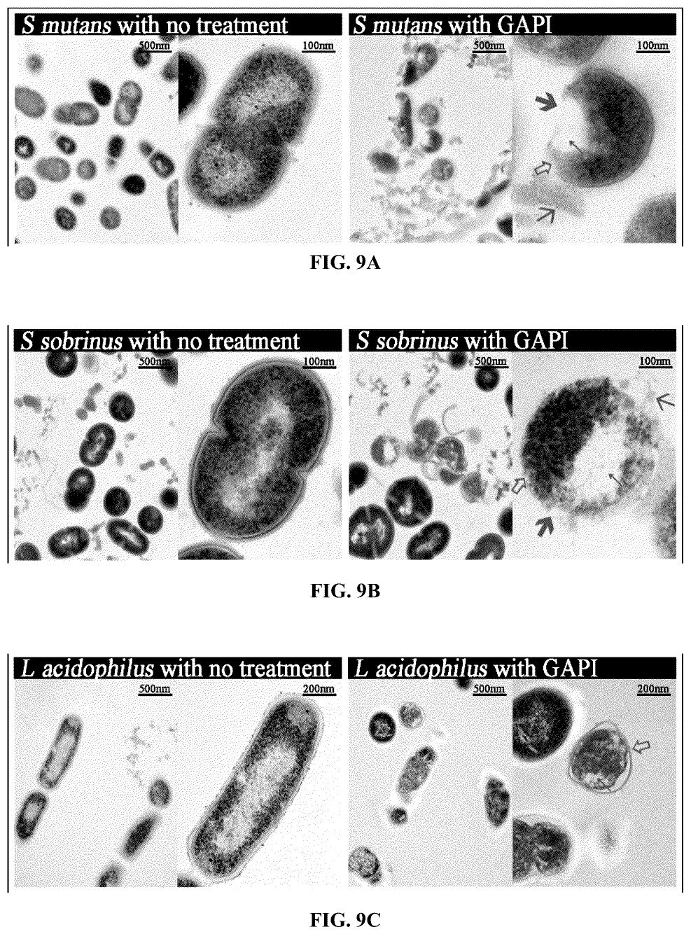

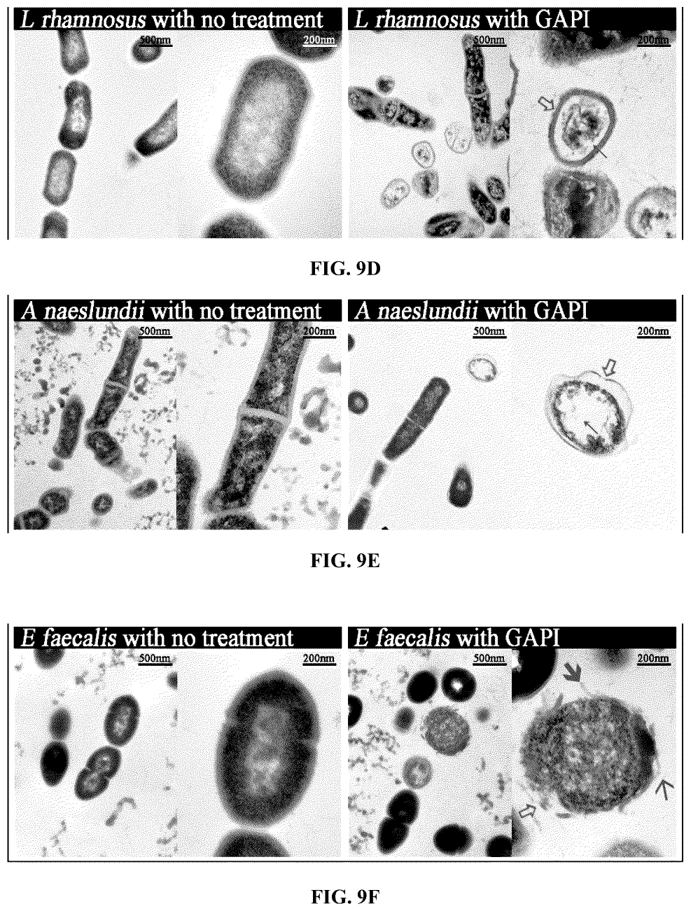

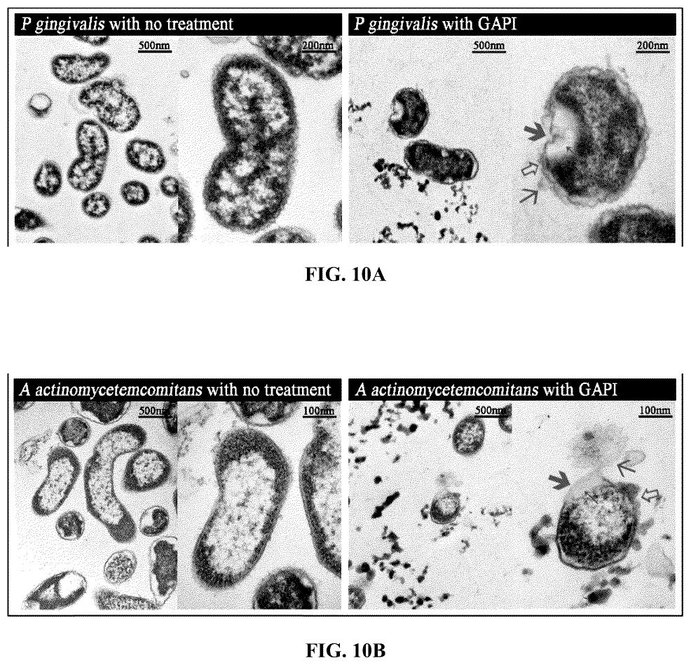

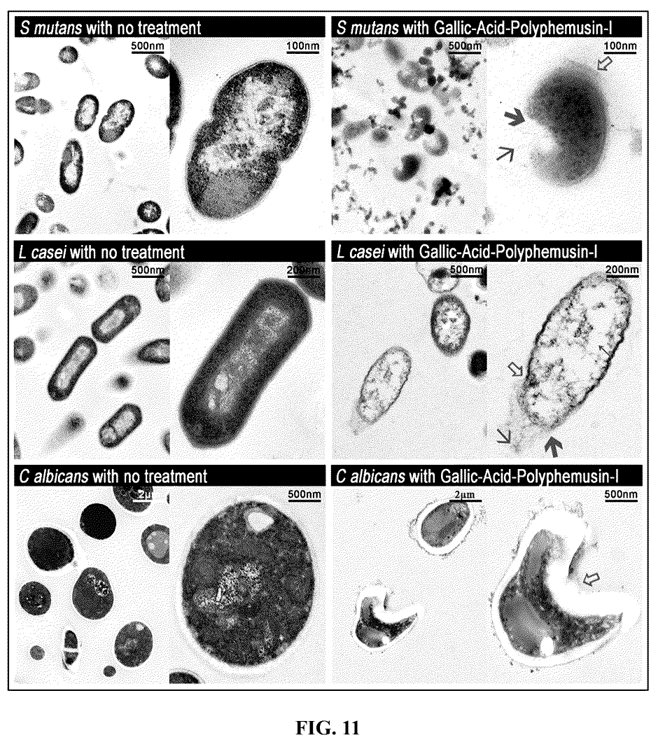

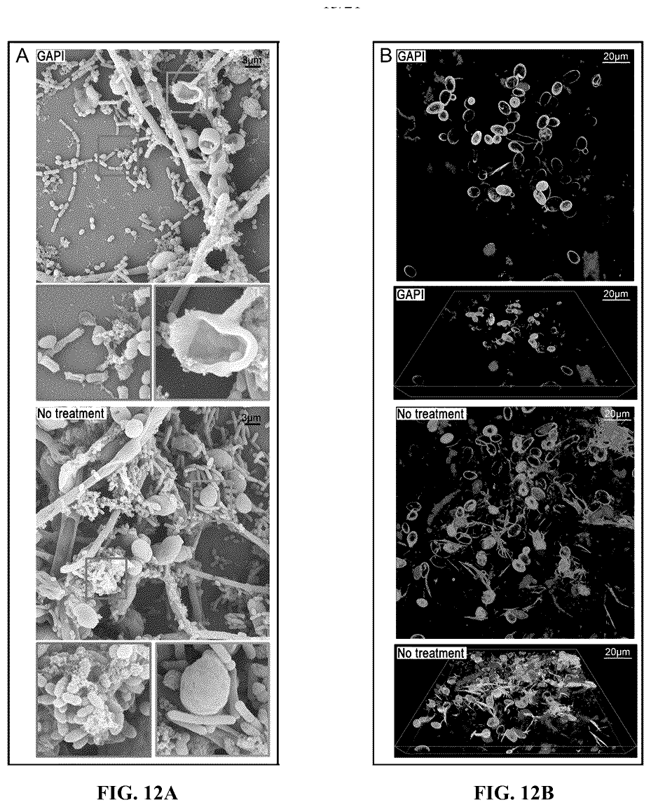

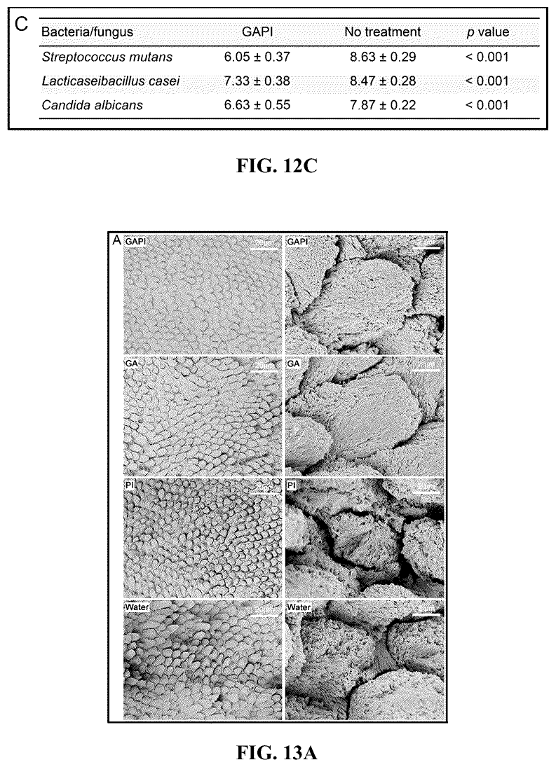

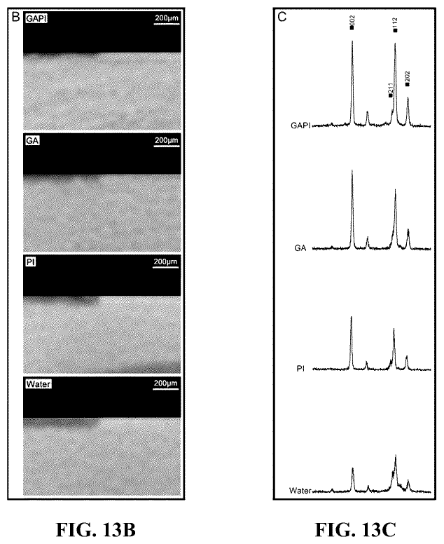

TEM was used to show bacterial morphology changes after GAPI treatment in order to further understand GAPI's mechanism (FIG. 9 and FIG. 10). The micrographs revealed that the GAPI disrupted the bacterial membrane, thus causing abnormal membrane curvature, irregular cell shapes, and intra-bacterial vacuolization, and inducing cytoplasmic components to escape from the microorganism. The mechanism of action begins with GAPI binding to bacteria and then interacting with the cytoplasmic membrane, thereby crossing the cytoplasmic membrane and damaging the membrane integrity. The damage to the integrity of the cell membrane is an important mechanism, by which antibacterial methods deactivate microorganisms. Furthermore, the TEM images indicated that GAPI could damage the bacterial cell structure, causing cytoplasmic content leakage.

### 5.2 Antibiofilm Properties

FIG. 9 shows the SEM micrographs and CLSM photographs of multiple-species biofilm treated with or without GAPI. GAPI treatment inhibited the forming of biofilm. In addition, the SEM micrographs showed that the cells in biofilm treated with GAPI lost their typical morphology (FIG. 9A). In contrast, a three-dimensional multiple-species biofilm formed in the control group. The CLSM photographs also demonstrated that GAPI obviously reduced the fluorescent signal of microorganisms in the biofilm. By the staining methods, Candida albicans (green) could be clearly distinguished from bacterial cells in the CLSM photographs (FIG. 9B). The quantitative analysis results of PMA-qPCR among different microorganisms are consistent. The log 10 cell counts of Streptococcus mutans treated with or without GAPI were 6.05±0.37 and 8.63±0.29 (p<0.001). The log 10 cell counts of Lacticaseibacillus casei treated with or without GAPI were 7.33±0.38 and 8.47±0.28 (p<0.001). And the log 10 cell counts of Candida albicans treated with or without GAPI were 6.63±0.55 and 7.87±0.22 (p<0.001) (FIG. 9C).

### 5.3 Remineralizing Effects

The SEM micrographs of the enamel surface showed a relatively smooth and homogenous prism pattern in the GAPI Group and the GA Group compared to other groups (FIG. 7A). In the PI Group and Water Group, the destruction of enamel prisms occurred in both the prism and inter-prism regions (FIG. 7A). Moreover, the enamel in the GAPI Group and GA Group had significantly higher calcium-to-phosphorus molar ratios than that in the PI Group and Water Group (Table 2).

The micro-CT figures show a consistent result compared to SEM micrographs: relatively undamaged enamel surface in the GAPI Group and the GA Group, but lesion surface in the PI Group and the Water Group (FIG. 7B). In addition, the enamel in the GAPI Group and the GA Group had significantly lower lesion depths and less mineral loss than those of the enamel in the PI Group and Water Group (Table 2).

TABLE 2
Mineral evaluations of enamel treated with Gallic-Acid-Polyphemusin-I (GAPI) peptide, Gallic
Acid (GA), Polyphemusin-I (PI) peptide, and deionized water (Water) (n = 8 per group)

| | GAPI ¹ | GA ² | PI ³ | Water ⁴ | p value | Bonferroni |
|---|---|---|---|---|---|---|
| Calcium to phosphate | 1.81 ± 0.05 | 1.82 ± 0.08 | 1.70 ± 0.06 | 1.71 ± 0.05 | =0.001 | 1, 2 > 3, 4 |
| molar ratio | | | | | | |
| Lesion depth (μm) | 64 ± 7 | 62 ± 12 | 96 ± 9 | 100 ± 10 | <0.001 | 1, 2 < 3, 4 |
| Mineral loss | 0.89 ± 0.20 | 0.88 ± 0.24 | 1.32 ± 0.26 | 1.28 ± 0.16 | <0.001 | 1, 2 < 3, 4 |
| (gHApcm⁻³) | | | | | | |

FIG. 7C shows the typical X-ray diffraction spectra of the four groups. The reflections of GAPI Group and PI Group were sharper compared with the other groups. The reflection peak of the four groups at full width and half maximum were 0.237°, 0.342°, 0.231° and 0.328°.

### 5.4 Discussion

This is the first study to develop an antimicrobial peptide with remineralizing properties by fusing gallic acid to polyphemusin-I. The results showed that the synthesized peptide GAPI inhibited the growth of cariogenic bacteria/fungi and remineralized early enamel caries.

Researchers have found that fusing antimicrobial peptides with functional sequences is an effective strategy for developing synthetic antimicrobial peptides with multifunction (Niu et al. 2021b). The synthesized GAPI consists of two domains: the remineralizing action domain, gallic acid and the antimicrobial action domain, polyphemusin-I. The role of Gallic acid is remineralizing function domain, because it contains pyrogallol groups which bind to calcium ions and accelerate hydroxyapatite regeneration (Cai et al. 2008; Prajatelistia et al. 2016; Zhou et al. 2012). Polyphemusin-I was selected as the antimicrobial domain because of its biocompatibility and antimicrobial activity (Zhang et al. 2019). In this study, the first step for synthesizing the novel peptide GAPI is the production of polyphemusin-I domain by bonding the carboxyl group (C terminus) of one amino acid to the amino group (N terminus) of another amino acid. Then, the gallic acid molecule was grafted to polyphemusin-I by bonding its carboxyl group to the N terminus amino group of polyphemusin-I. The molecular weight of synthesized peptide was 152.92 kD more than that of polyphemusin-I. It is equal to the molecular weight of gallic acid minus water molecule due to polymerization. The difference of molecular weight is consistent with the synthesis procedure mentioned above. The synthesis method of GAPI used in this study is the standard solid-phase synthesis method. Although the solid-phase peptide synthesis method has limited yields, it is simpler to perform purification procedures than solution-phase peptide synthesis.

The secondary structure of peptides was measured using circular dichroism spectroscopy. Polyphemusin-I display a β-turn structure which means the connection of antiparallel β-sheets exists in polyphemusin-I. The β-sheet antimicrobial peptides can bind to the lipid bilayer of microbial membranes, electrostatically interacts with the bacterial cell and translocate across the lipid bilayer. These activities cause disruption of the cytoplasmic membrane which is the possible antimicrobial mechanism of peptides (Edwards et al. 2016). The β-sheet is indispensable for the antimicrobial activity of polyphemusin-I. Previous studies have demonstrated that interrupting of the β-sheet structure of polyphemusin-I reduces the antimicrobial activity and eliminates the ability to translocate across membranes (Powers et al. 2004). Therefore, it is essential to confirm the secondary structure of synthetic antimicrobial peptides GAPI (Niu et al. 2021b). In our study, the percentages of the β-sheet of the GAPI were lower than those of polyphemusin-I, which explains that the antimicrobial activity of GAPI is slightly less than polyphemusin-I alone. However, this minor reduction is acceptable because we confer the novel peptide's remineralization ability in this study.

The biocompatibility and stability of the synthesized peptide is vitally important for further use in dental clinic. This study showed that the concentration of GAPI starting to affect the proliferation of human gingival fibroblast is higher than its minimum bactericidal/fungicidal concentration against cariogenic species. Thus, the safety of GAPI for dental use is confirmed. Human saliva maintains a neutral condition because of its buffering activity and continual replenishment. This study showed GAPI was stable for at least 60 min in saliva, which should be plenty of time to exert its biological effects.

In this study, we assessed the antimicrobial effect of peptides against bacteria/fungi under planktonic conditions. Three acidogenic and cariogenic microorganisms were employed: Streptococcus mutans, Lacticaseibacillus casei and Candida albicans. Streptococcus mutans is a pioneer species in caries development. It can adhere to the acquired pellicle and other plaque bacteria to establish a cariogenic biofilm (Mei et al. 2013). Lacticaseibacillus casei is frequently detected in caries lesions and is associated with dental caries (Caufield et al. 2015; Reis et al. 2021). Candida albicans also contributes to caries progression or recurrence (Koo and Bowen 2014; Krzysciak et al. 2017). The MIC and MBC/MFC tests are standard tests in laboratory studies on antimicrobial peptides (Niu et al. 2021b). The results showed that GAPI can effectively inhibit planktonic Streptococcus mutans, Lacticaseibacillus casei and Candida albicans.

In addition, this study analyzed the cell morphology of bacteria/fungi treated with GAPI to investigate the potential mechanism of antimicrobial activity. We found that the interaction between peptide and microbial membrane induces negative membrane curvature strain and causes irregular cell shape. In this study, abnormal membrane curvature was the most typical defect. Moreover, it is likely that the peptide translocates across the lipid bilayer to act on intracellular targets to form transparent cytoplasmic zones. These are consistent with descriptions of the antimicrobial mechanisms of the peptides in other studies (Yeaman and Yount 2003; Zhang et al. 2019). In this study, we also found that a thorough disruption of the cytoplasmic membrane occurred in Streptococcus mutans cells, which can cause leakage of cytoplasmic content. Previous studies indicated that β-sheet peptides, such as polyphemusin-I, are not likely to induce pore formation or significant membrane damage (Powers et al. 2006). However, the different membrane-active mechanisms should not be mutually exclusive; they may be mutually causal (Lv et al. 2014). In addition, this is the first study to investigate the damage of a peptide derived from polyphemusin-I on Streptococcus mutans by using TEM. These findings brought new knowledge on the mechanism of antimicrobial peptides.

This study used Streptococcus mutans, Lacticaseibacillus casei and Candida albicans to develop a multiple-species cariogenic biofilm for assessing the anti-biofilm properties of GAPI. This study used ConA to examine Candida albicans and the bacteria, which could be clearly distinguished under CLSM (Yassin et al. 2016). In addition, the quantitative analysis of microorganisms was conducted by PMA-qPCR. The usual qPCR approach detects total DNA from live and dead cells (Alvarez et al. 2013). A real-time PCR method combined with PMA was conducted (Takahashi et al. 2011) because discriminating live and dead cells is essential to assess the activity of antimicrobial peptides. The SEM, CLSM and PMA-qPCR results jointly demonstrated the anti-biofilm effect of GAPI on multiple-species biofilms. A multiple-species cariogenic biofilm is superior compared to mono-species biofilm for studying caries activity (Wang et al. 2020). However, the results cannot be extrapolated because around 700 species have been shown to be capable of colonizing the oral cavity (Mei et al. 2013).

A chemical pH cycling model was used to investigate the remineralizing effect of GAPI in this study. The pH cycling model consists of demineralization and remineralization procedures. This model uses a chemical acid challenge to simulate a high-risk caries situation. After the pH cycling procedure, a non-destructive measurement, Micro-CT, was used first to assess the enamel blocks. The advantages of Micro-CT include avoiding the need for a complicated sample preparation. The samples can still be used for other tests after scanning and the collected data could be transferred to quantifiable data (lesion depth, mineral loss) for statistical analysis. Furthermore, SEM-EDS was used to analyze the elemental composition of the enamel block's surface. Then, the Ca/P molar ratio could be calculated. A higher Ca/P molar ratio indicated a lower solubility of calcium phosphate. The abovementioned quantifiable indexes are sufficient to illustrate the remineralizing agent's effects.

For the descriptive analysis, the scanned data of the Micro-CT could be restructured to observe the integrity of the enamel surface. An SEM micrograph further confirmed the enamel surface structure. Sound enamel had a smooth surface in the SEM micrograph. At the beginning of demineralization, the prism patterns resembled well-organized fish scales due to the enamel crystal dissolution from the prisms' edge. After prolonged demineralization, the dissolution of enamel crystal occurred in both prisms and inter-prism regions. The enamel surface was uneven and the prism patterns were disordered. Furthermore, X-ray diffraction analysis was used to analyze the crystallization of hydroxyapatite in the enamel blocks. The full width at half-maximum value indicates the crystallite size (Cao et al. 2014). In this study, we observed consistent results among the statistical and descriptive analyses which illustrate the remineralizing properties of GAPI.

### 5.5 Conclusion

A novel biocompatible anti-caries Gallic-Acid-Polyphemusin-I (GAPI) peptide was developed by fusing Gallic acid to Polyphemusin-I. This novel peptide inhibited the growth of cariogenic bacteria and fungi. It can also remineralized early enamel caries. The promising effects of our synthesized peptides demonstrated its potential to act as an anti-caries agent for clinical use.

### 5.6 Antimicrobial Peptide (or AMP)

The composition disclosed herein comprises one or more AMP at a level where upon directed use, they promote the benefit sought by the subject (e.g., inhibition of S. mutans, decrease in caries formation, etc.) without detriment to the oral surface.

In certain embodiments, the antimicrobial peptide whose amino acid sequence is RRWCFRVCYRGFCYRKCR (SEQ ID NO:10). In certain embodiments, the antimicrobial peptide comprises a plurality of amino acid peptides. In certain embodiments, multiple amino acid peptides may be joined by linkers.

In various embodiments the antimicrobial peptides are attached to one or more antimicrobial peptides via one or more linking agents. In certain embodiments, the antimicrobial peptides can be conjugated via a single linking agent or multiple linking agents. For example, the antimicrobial peptides can be conjugated via a single multifunctional (e.g., bi-, tri-, or tetra-) linking agent or a pair of complementary linking agents. In another embodiment, the antimicrobial peptides are conjugated via two, three, or more linking agents. Suitable linking agents include, but are not limited to, e.g., functional groups, affinity agents, stabilizing groups, and combinations thereof.

In certain embodiments the linking agent is or comprises a functional group. Functional groups include monofunctional linkers comprising a reactive group as well as multifunctional crosslinkers comprising two or more reactive groups capable of forming a bond with two or more different functional targets (e.g., labels, proteins, macromolecules, semiconductor nanocrystals, or substrate). In some preferred embodiments, the multifunctional crosslinkers are heterobifunctional crosslinkers comprising two or more different reactive groups.

It will be noted that in various embodiments, the AMP comprises a peptide that ranges in length from 5-10 amino acid, or from 10-15 amino acids, or from 15-18 amino acids, or from 18-36 amino acids, from 36-54 amino acids, from 54-72 amino acids. Similarly, in various embodiments, the antimicrobial peptide comprises a peptide that ranges in length from 5 amino acids, or from 6 amino acids, or from 7 amino acids, or from 8 amino acids, or from 9 amino acids, or from 10 amino acids up to about 100 amino acids, or up to about 80 amino acids, or up to about 60 amino acids, or up to about 50 amino acids, or up to about 40 amino acids, or up to about 30 amino acids, or up to about 20 amino acids.

### 5.7 Additional Agents

The composition of the present disclosure can additionally include one or more active agents. In certain embodiments, the active agents comprise a positively charged compound that is antimicrobial and/or that promotes remineralization. In certain embodiments, the composition additionally includes a fluoride. In certain embodiments, the additional active agents can comprise one or more remineralization agents. In certain embodiments, antibacterial or antiseptic agents such as bleach, or antiseptic agents with general positive charge can be incorporated into the composition.

### 5.8 Lipids and Liposomes

In certain embodiments, the AMP described herein are attached to one or more microparticles or nanoparticles that can be loaded with an effector agent (e.g., a pharmaceutical, a label, etc.). In certain embodiments, the microparticles or nanoparticles are lipidic particles. Lipidic particles are microparticles or nanoparticles that include at least one lipid component forming a condensed lipid phase. Typically, a lipidic nanoparticle has preponderance of lipids in its composition. Various condensed lipid phases include solid amorphous or true crystalline phases; isomorphic liquid phases (droplets); and various hydrated mesomorphic oriented lipid phases such as liquid crystalline and pseudocrystalline bilayer phases (L-alpha, L-beta, P-beta, Lc), interdigitated bilayer phases, and nonlamellar phases (see, e.g., The Structure of Biological Membranes, ed. by P. Yeagle, CRC Press, Boca Raton, Fla., 1991). Lipidic microparticles include, but are not limited to a liposome, a lipid-nucleic acid complex, a lipid-drug complex, a lipid-label complex, a solid lipid particle, a microemulsion droplet, and the like. Methods of making and using these types of lipidic microparticles and nanoparticles, as well as attachment of affinity moieties, e.g., antibodies, to them are known in the art.

A liposome is generally defined as a particle comprising one or more lipid bilayers enclosing an interior, typically an aqueous interior. Thus, a liposome is often a vesicle formed by a bilayer lipid membrane. There are many methods for the preparation of liposomes. Some of them are used to prepare small vesicles (d<0.05 micrometer), some for larger vesicles (d>0.05 micrometer). Some are used to prepare multilamellar vesicles, some for unilamellar ones. Methods for liposome preparation are exhaustively described in several review articles such as Szoka and Papahadjopoulos (1980) Ann. Rev. Biophys. Bioeng., 9: 467, Deamer and Uster (1983) Pp. 27-51 In: Liposomes, ed. M. J. Ostro, Marcel Dekker, New York, and the like.

In various embodiments, the liposomes include a surface coating of a hydrophilic polymer chain. “Surface-coating” refers to the coating of any hydrophilic polymer on the surface of liposomes. The hydrophilic polymer is included in the liposome by including in the liposome composition one or more vesicle-forming lipids derivatized with a hydrophilic polymer chain. In certain embodiments, vesicle-forming lipids with diacyl chains, such as phospholipids, are preferred. One illustrative phospholipid is phosphatidylethanolamine (PE), which contains a reactive amino group convenient for coupling to the activated polymers. One illustrative PE is distearoyl PE (DSPE). Another example is non-phospholipid double chain amphiphilic lipids, such as diacyl- or dialkylglycerols, derivatized with a hydrophilic polymer chain.

In certain embodiments, a hydrophilic polymer for use in coupling to a vesicle forming lipid is polyethyleneglycol (PEG), preferably as a PEG chain having a molecular weight between 1,000-10,000 Daltons, more preferably between 1,000-5,000 Daltons, most preferably between 2,000-5,000 Daltons. Methoxy or ethoxy-capped analogues of PEG are also useful hydrophilic polymers, commercially available in a variety of polymer sizes, e.g., 120-20,000 Daltons.

Other hydrophilic polymers that can be suitable include, but are not limited to polylactic acid, polyglycolic acid, polyvinylpyrrolidone, polymethyloxazoline, polyethyloxazoline, polyhydroxypropyl methacrylamide, polymethacrylamide, polydimethylacrylamide, and derivatized celluloses, such as hydroxymethylcellulose or hydroxyethylcellulose.

### 5.9 Administration and Formulations

In certain embodiments, the AMPs are administered to a mammal in need thereof, to a cell, to a tissue, to a composition (e.g., a food), etc.). In various embodiments, the compositions can be administered to detect and/or locate, and/or quantify the presence of particular microorganisms, microorganism populations, biofilms comprising particular microorganisms, and the like. In various embodiments, the compositions can be administered to inhibit particular microorganisms, microorganism populations, biofilms comprising particular microorganisms, and the like.

The AMPs can be administered in the “native” form or, if desired, in the form of salts, esters, amides, prodrugs, derivatives, and the like, provided the salt, ester, amide, prodrug or derivative is suitable pharmacologically, i.e., effective in the present method(s). Salts, esters, amides, prodrugs and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

Methods of formulating such derivatives are known to those of skill in the art. For example, the disulfide salts of a number of delivery agents are described in PCT Publication WO 2000/059863 which is incorporated herein by reference. Similarly, acid salts of therapeutic peptides, peptoids, or other mimetics, and can be prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt can be reconverted to the free base by treatment with a suitable base. Certain particularly preferred acid addition salts of the active agents herein include halide salts, such as may be prepared using hydrochloric or hydrobromic acids. Conversely, preparation of basic salts of the active agents of this disclosure are prepared in a similar manner using a pharmaceutically acceptable base such as sodium hydroxide, potassium hydroxide, ammonium hydroxide, calcium hydroxide, trimethylamine, or the like. In certain embodiments basic salts include alkali metal salts, e.g., the sodium salt, and copper salts.

In various embodiments, the active agents identified herein are useful for parenteral, topical, oral, nasal (or otherwise inhaled), rectal, or local administration, such as by aerosol or transdermally, for detection and/or quantification, and or localization, and/or prophylactic and/or therapeutic treatment of infection (e.g., microbial infection). The compositions can be administered in a variety of unit dosage forms depending upon the method of administration. Suitable unit dosage forms, include, but are not limited to powders, tablets, pills, capsules, lozenges, suppositories, patches, nasal sprays, injectable, implantable sustained-release formulations, lipid complexes, etc.

The active agents (e.g., antimicrobial peptides) described herein can also be combined with a pharmaceutically acceptable carrier (excipient) to form a pharmacological composition. In certain embodiments, pharmaceutically acceptable carriers include those approved by a regulatory agency of the Federal or a state government or listed in the U.S. Pharmacopeia or other generally recognized pharmacopeia for use in/on animals, and more particularly in/on humans. A “carrier” refers to, for example, a diluent, adjuvant, excipient, auxiliary agent or vehicle with which an active agent of the present disclosure is administered.

Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

Other physiologically acceptable compounds, particularly of use in the preparation of tablets, capsules, gel caps, and the like include, but are not limited to binders, diluent/fillers, disentegrants, lubricants, suspending agents, and the like.

In certain embodiments, to manufacture an oral dosage form (e.g., a tablet), an excipient (e.g., lactose, sucrose, starch, mannitol, etc.), an optional disintegrator (e.g. calcium carbonate, carboxymethylcellulose calcium, sodium starch glycollate, crospovidone etc.), a binder (e.g. alpha-starch, gum arabic, microcrystalline cellulose, carboxymethylcellulose, polyvinylpyrrolidone, hydroxypropylcellulose, cyclodextrin, etc.), and an optional lubricant (e.g., talc, magnesium stearate, polyethylene glycol 6000, etc.), for instance, are added to the active component or components (e.g., active peptide) and the resulting composition is compressed. Where necessary the compressed product is coated, e.g., known methods for masking the taste or sustained release. Suitable coating materials include, but are not limited to ethyl-cellulose, hydroxymethylcellulose, polyoxyethylene glycol, cellulose acetate phthalate, hydroxypropylmethylcellulose phthalate, and Eudragit (Rohm & Haas, Germany; methacrylic-acrylic copolymer).

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s).

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain therapeutic applications, the compositions of this disclosure are administered, e.g., topically administered or administered to the oral or nasal cavity, to a patient suffering from infection or at risk for infection or prophylactically to prevent dental caries or other pathologies of the teeth or oral mucosa characterized by microbial infection in an amount sufficient to prevent and/or cure and/or at least partially prevent or arrest the disease and/or its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the active agents of the formulations of this disclosure to effectively treat (ameliorate one or more symptoms in) the patient.

The concentration of active agent(s) can vary widely, and will be selected primarily based on activity of the active ingredient(s), body weight and the like in accordance with the particular mode of administration selected and the patient's needs. Concentrations, however, will typically be selected to provide dosages ranging from about 0.1 or 1 mg/kg/day to about 50 mg/kg/day and sometimes higher. Typical dosages range from about 3 mg/kg/day to about 3.5 mg/kg/day, preferably from about 3.5 mg/kg/day to about 7.2 mg/kg/day, more preferably from about 7.2 mg/kg/day to about 11.0 mg/kg/day, and most preferably from about 11.0 mg/kg/day to about 15.0 mg/kg/day. In certain preferred embodiments, dosages range from about 10 mg/kg/day to about 50 mg/kg/day. In certain embodiments, dosages range from about 20 mg to about 50 mg given orally twice daily. It will be appreciated that such dosages may be varied to optimize a therapeutic and/or phophylactic regimen in a particular subject or group of subjects.

In certain embodiments, the active agents of this disclosure are administered to the oral cavity. This is readily accomplished by the use of lozenges, aerosol sprays, mouthwash, coated swabs, patches and the like.

### 6. EXAMPLES

### Materials and Methods

### 6.1 Synthesis and Characterization

A novel antimicrobial peptide with remineralizing properties was developed by fusing gallic acid to the N-terminus of polyphemusin-I (RRWCFRVCYRGFCYRKCR)(SEQ ID NO:10). The disulfide bonds for C4 and C17, C8 and C13 are needed. Thus, we used the Trityl (Trt) group to protect the cysteine residue for C4 and C17, whereas the acetamidomethyl (Acm) group was used to protect the cysteine residue for C8 and C13. After peptide synthesis using solid-phase peptide synthesis (SPPS) method, Trifluoroacetic acid (TFA) was used to perform deprotection of the Trt group on C4 and C17. Then, air oxidation method was used to form disulfide bonds on C4 and C17. After that, dithiothreitol (DTT) was used to perform deprotection of the Acm group on C8 and C13. The same methods were used to form another disulfide bond on C8 and C13. The synthesis of β-sheet PI with the fixed positions of disulfide bonds between C4 and C17, C8 and C13 was finished. Then, the SPPS method was used to graft gallic acid to the peptide. Polyphemusin-I and GAPI were synthesized using the standard fluorenylmethoxycarbonyl solid-phase synthesis method (Cao et al. 2014). Then, the purity and molecular weight of peptides were determined using high-performance liquid chromatography (HPLC) and mass spectrometry, respectively. The secondary structure of peptides was investigated using circular dichroism spectroscopy (Chirascan, AppliedPhotophysics, United Kingdom) and modelled using the software CDPro (Jasco, Corp, Tokyo, Japan) (Niu et al. 2021d). The GAPI powder was dissolved in sterile deionized water to a specific concentration for study and was stored at −20° C.

### 6.2 Stability in Human Saliva

The stability of peptides in human saliva was investigated by testing the remaining peptide concentration via HPLC system (Waters Pacific Pte Ltd, Singapore). The saliva with peptides was mixed and incubated for 0, 15, 30, 45 and 60 min. The test was processed following a previous experimental protocol (Huang et al. 2016).

### 6.3 Biocompatibility

The biocompatibility of GAPI was evaluated by cytotoxicity assay using Cell Counting Kit-8 Assay (CCK-8, Apexbio, MA, USA) (Zhang et al. 2019). Human gingival fibroblast (HGF-1, Otwo Biotech Inc., China) cells (1×105/mL) were cultured and treated with GAPI. The cells cultured with the only medium were negative controls. The optical density values indicating the number of viable cells was measured at 450 nm after 24 h of incubation.

### 6.4 Microorganisms

Common oral pathogenic bacterial strains were selected for this study. Streptococcus mutans ATCC 35668, Streptococcus sobrinus ATCC 33478, Lactobacillus acidophilus ATCC 9224, Lactobacillus rhamnosus ATCC10863, Actinomyces naeslundii ATCC 12104, Enterococcus faecalis ATCC 29212, Porphyromonas gingivalis ATCC 33277, and Actinobacillus actinomycetemcomitans ATCC 29523. All the strains were cultured anaerobically.

### 6.5 Antimicrobial Properties

The antimicrobial properties of synthesized peptides against bacteria and fungi were evaluated using minimum inhibitory concentration (MIC) and minimum bactericidal/fungicidal concentration (MBC/MFC) tests (Wiegand et al. 2008). The tested species include Streptococcus mutans, UA159; Lacticaseibacillus casei, American Type Culture Collection (ATCC) 334; and Candida albicans, ATCC 90028. A 10 μL aliquot of bacterial culture (106 CFU/mL) in brain heart infusion (BHI) broth was added to 100 μL serial two-fold dilutions of experimental solutions. The optical density values at 660 nm were measured after 18 h of incubation. To determine the MBC, 10 μL of bacterial culture was taken from the well of MIC, and wells before and after the well of MIC, and transferred to blood agar. The test protocol for fungi was similar: the culture medium was a Roswell Park Memorial Institute (RPMI) 1640 Medium, the absorbance was measured at 520 nm, and Sabouraud dextrose agar was used for the MFC test. Chlorhexidine and medium were used as the positive and negative controls.

### 6.6 μMIC AND MBC

Brain heart infusion (BHI) medium was used for culture of S. mutans, S. sobrinus, L. acidophilus, L. rhamnosus, A. naeslundii, E. faecalis, and A. actinomycetemcomitans, whereas p.g. broth was used for culture of Porphyromonas gingivalis. The standard dilution method in a 96-well microplate was conducted in order to evaluate the antimicrobial efficacy of GAPI. Each well was filled with 100 L GAPI dilutions. In addition, serial twofold dilutions in concentrations ranging from 1280 μM to 1.25 μM were prepared. A 10 μL bacterial culture (106 CFU/mL) was added. Chlorhexidine was used as positive control, and medium was used as negative control. The plates were then anaerobically incubated at 37° C. for 24 h. The absorbance was measured at a wavelength of 660 nm in order to analyze the growth of microorganisms. The MIC value was defined as the lowest concentration at which no visible growth was seen in the clear well. After the MIC determination, 10 μL fluid from each well, which showed no visible bacterial growth, was pipetted and seeded on blood agar, which were then put into an anaerobic incubator at 37° C. for 48 h. The MBC endpoint was the lowest concentration at which 99.9% of the bacterial population was killed, which thus means the absence of bacteria. The morphology of the bacteria and fungi treated with synthesized peptides was analysed using transmission electron microscopy (TEM) (Philips CM100, Philips/FEI Corporation, Eindhoven, Holland). The sample was prepared following a previous study (Niu et al. 2021d). Copper grids were used to contain the semi-thin sections of bacteria/fungi for examination.

### 6.7. Morphology of the Microorganisms

Bacteria morphology was observed using a transmission electron microscope (TEM, Philips CM100). GAPI was added to a bacterial culture of 108 CFU/mL, and the bacteria were harvested after incubating at 37° C. for 18 h. The semi-thin sections of cell were contained in grids and examined with the TEM.

### 6.8 Antibiofilm Properties

A multiple-species cariogenic biofilm consisting of Streptococcus mutans, Lacticaseibacillus casei and Candida albicans was cultured to assess the anti-biofilm properties of GAPI. The culture medium was modified-artificial saliva as described in a previous protocol (Yassin et al. 2016). The biofilm was cultured on glass slides in 24-well plates with 1×10⁷ Streptococcus mutans, 1×10⁷ Lacticaseibacillus casei and 1×10⁵ Candida albicans for 48 h. The biofilm was then treated with GAPI. The biofilm with no treatment was the negative control. After another 48-h incubation, the biofilm was collected for sequent assessments. The architecture of the multiple-species biofilm was assessed using scanning electron microscopy (SEM) and confocal laser scanning microscopy (CLSM). The sample was prepared with our published protocol (Niu et al. 2021d). The growth kinetics of the multiple-species biofilm were analyzed using propidium monoazide-quantitative polymerase chain reaction (PMA-qPCR) (Loozen et al. 2011). Biofilm was suspended in phosphate-buffered solution. The suspension was treated with propidium monoazide (PMA) (Biotium, San Francisco, USA) and exposed to halogen light photoactivation (PMA-Lite™, Biotium, San Francisco, USA). The brightness (luminosity) of the light device is 600-800 millicandela and its wavelength is 465-475 nm. Then deoxyribonucleic acid (DNA) was extracted from PMA-treated biofilm suspension using QIAamp DNA Mini Kit (QIAGEN, Dusseldorf, Germany). The oligonucleotide primers and TaqMan probes were used to quantify multiple-species cariogenic biofilm species: SMUT-forward(F), SMUT-reverse(R) and SMUT-probe(P) for Streptococcus mutans, Lc-F, Lc-R, Lc-P for Lactobacillus casei and Ca—F, Ca—R and Ca-P for Candida albicans (6.8.1). Taqman reactions contained 1 μl each primer and probe, 10 μl master mix (Applied Biosystems, Waltham, USA), 2 μl sterile water and 5 μl sample DNA. The thermocycling was conducted in a StepOnePlus real-time PCR system (Applied Biosystems, Waltham, USA) under the following conditions: an initial 2 min at 50° C., followed by a denaturation step for 10 min at 95° C., followed by 50 cycles of 95° C. for 15 s and 58° C. for 60 s. A standard curve was constructed using a series of known concentrations of microorganism cells for each species.

Biofilm was suspended in phosphate-buffered solution. The suspension was treated with propidium monoazide (PMA) (Biotium, San Francisco, USA) and exposed to halogen light photoactivation (PMA-Lite™, Biotium, San Francisco, USA). The brightness (luminosity) of the light device is 600-800 millicandela and its wavelength is 465-475 nm. Then deoxyribonucleic acid (DNA) was extracted from PMA-treated biofilm suspension using QIAamp DNA Mini Kit (QIAGEN, Dusseldorf, Germany). The oligonucleotide primers and TaqMan probes were used to quantify multiple-species cariogenic biofilm species: SMUT-forward(F), SMUT-reverse(R) and SMUT-probe(P) for Streptococcus mutans, Lc-F, Lc-R, Lc-P for Lactobacillus casei and Ca—F, Ca—R and Ca-P for Candida albicans (Table 3). Tagman reactions contained 1 μl each primer and probe, 10 μl master mix (Applied Biosystems, Waltham, USA), 2 μl sterile water and 5 μl sample DNA. The thermocycling was conducted in a StepOnePlus real-time PCR system (Applied Biosystems, Waltham, USA) under the following conditions: an initial 2 min at 50° C., followed by a denaturation step for 10 min at 95° C., followed by 50 cycles of 95° C. for 15 s and 58° C. for 60 s. A standard curve was constructed using a series of known concentrations of microorganism cells for each species.

TABLE 3
List of the oligonucleotide primers and TaqMan
probes for the propidium monoazide-quantitative
polymerase chain reaction test

| Name | Sequence |
|---|---|
| Ca-F | 5′-GTGAATCATCGAATCTTTGAAC-3′ SEQ ID NO: 1 |
| Ca-R | 5′-TCCTCCGCTTATTGATATGC3′ SEQ ID NO: 2 |
| Ca-P | 6FAM-ATTGCTTGCGGCGGTAACGTCC-TAMRA SEQ ID |
| | NO: 3 |
| Lc-F | 5′-AGGCGGCTGTCTGGTCTGTA-3′ SEQ ID NO: 4 |
| Lc-R | 5′-CCTGTTCGCTACCCATGCTT-3′ SEQ ID NO: 5 |
| Lc-P | 6FAM-TGACGCTGAGGCTC-TAMRA SEQ ID NO: 6 |
| SMUT-F | 5′-GCCTACAGCTCAGAGATGCTATTCT-3′ SEQ ID NO: |
| | 7 |
| SMUT-R | 5′-GCCATACACCACTCATGAATTGA-3′ SEQ ID NO: 8 |
| SMUT-P | 6FAM- TGGAAATGACGGTCGCCGTTATGAA-TAMRA SEQ |
| | ID NO: 9 |

### 6.9 Remineralizing Effects

Ten enamel slices were cut from extracted sound human molars. Each slice was separated into four blocks. A total of 40 enamel blocks were prepared. Then the surface of the enamel block was half-covered by an acid-resistant varnish. The exposed half was the experimental surface, and the varnished half was the internal control. Before use, all the enamel blocks were sterilized via autoclave (Niu et al. 2021d; Yu et al. 2018).

The pH-cycling procedure (16 h-demineralization at pH 4.5 and 8 h-remineralization at pH 7.0) was performed to enamel blocks for 8 days. The formula for demineralizing/remineralizing solution was employed from a previous study (Yu et al. 2018). The enamel blocks from the same enamel slice were treated twice daily with 160 μM GAPI (GAPI Group), 160 μM gallic acid (GA Group), 160 μM polyphemusin-I (PI Group), and sterile deionized water (Water Group). All enamel blocks after pH cycling were involved in sequent assessments.

The structure of the enamel surface was examined using SEM (n=8 per group) (Niu et al. 2021d). The elemental analysis of enamel surface was conducted using affiliated energy-dispersive X-ray spectroscopy (EDS). The calcium to phosphorus molar ratio was calculated (Niu et al. 2022). The mineral loss and lesion depth on the enamel blocks were assessed using microcomputed tomography (Micro-CT) (1076, SkyScan, Antwerp, Belgium) (n=8 per group) (Niu et al. 2022). The diffraction patterns of the enamel blocks were evaluated using X-ray diffraction analysis (XRD) (Rigaku SmartLab 9 kW, Bruker AXS GmbH, Karlsruhe, Germany) (n=2 per group) (Yu et al. 2018).

### 6.10 Sample Size Calculation and Statistical Analyses

The mean lesion depth of the experimental group was around 60 m in our pilot study. Assuming that the common standard deviation was 20 m with power at 0.90 and α=0.05, the sample size was at least six in each group. Thus, we set the sample size at n=8. The data was analyzed using SPSS Statistics 20 (IBM Corporation, Somers, NY, USA). The Shapiro-Wilk test was employed for testing normal distribution. The log 10 cell count of microorganisms was compared using t-test. The one-way analysis of variance and Bonferroni post hoc test were used to determine the differences in CCK-8 test, lesion depth, mineral loss and calcium to phosphorus molar ratios.

### 7. EXAMPLE

### 7.1 Materials and Methods

### 7.1. Synthesis of Peptide

GAPI was synthesized using standard fluorenylmethoxycarbonyl synthesis through conventional solid-phase peptide synthesis. The GAPI powder was dissolved in sterile deionized water and stored at −20° C.

### 7.2. Preparation of Dentine Blocks with Artificial Carious Lesions

Dentine slices, 2 mm thick, were cut from sound extracted third molars. Ultra-fine 4000-grit sandpaper was used to polish these slices. Slices with defects were eliminated based on a stereomicroscope observation. A total of forty dentine slices were selected. Each slice was separated into two blocks and assigned to two groups. Nail varnish was used to coat half of the blocks as an internal control [14]. S. mutans American Type Culture Collection 35668 was utilized in the present study. All the dentine blocks were placed into S. mutans culture (108 cells/mL) in a brain-heart infusion (BHI) broth containing 5% glucose to generate artificial caries. After a 3-day anaerobic culture at 37° C., the depth of the caries lesion surface was around 96 μm, which was observed using micro-computed tomography (micro-CT).

### 7.3. Experimental Treatment

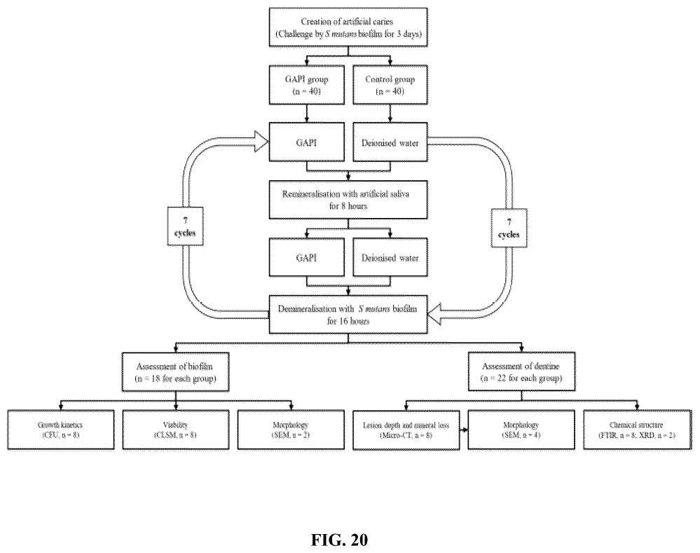

Two blocks from the same slice were assigned to the GAPI group (160 μM GAPI) and control group (deionized water) separately. All the blocks underwent a biochemical model involving chemical remineralization and an S. mutans biofilm challenge for seven days. The remineralization solution (pH 7.0) consisted of 1.5 mm calcium chloride, 150 mm potassium chloride, 0.9 mm potassium dihydrogen phosphate, and 20 mm 4-(2-hydroxyethyl)-1-piperazineethanesulfonic acid. The cycling process included 8 h of immersion in the remineralization solution and 16 h of immersion in an S. mutans biofilm medium. FIG. 20 shows the research protocol of this study.

### 7.4. Antibiofilm Effect of GAPI Peptide

### 7.4.1. Biofilm Viability

CLSM (Fluoview FV 1000, Olympus, Tokyo, Japan) was utilized to capture images to assess the S. mutans biofilm viability (n=8 for each group). Two fluorescent probes were used to label the bacteria (LIVE/DEAD BacLight Bacterial viability kit, Molecular Probes, Eugene, OR, USA). Among these probes, the propidium iodide probe stained the dead bacterial cells red, and the SYTO-9 probe stained the live bacterial cells green. The red-green ratio represented the dead-live ratio calculated using Image J version 1.53 t (National Institute of Health, Bethesda, MD, USA).

### 7.4.2. Biofilm Kinetics

To determine the S. mutans biofilm growth kinetics, the colony-forming units (CFUs) were counted (n=8 for each group). The dentin blocks that underwent biochemical cycles were slightly rinsed with a phosphate-buffered solution. Then, the blocks were shaken in a vortex machine to collect the bacterial cells in a 1 mL BHI broth. Subsequently, a ten-fold serial dilution of the bacterial solution was prepared, and 10 μL of each dilution was plated on blood agar. All the plates were cultured anaerobically for 48 h at 37° C. before the CFUs were counted. The log CFU was calculated for a statistical analysis.

### 7.4.3. Biofilm Morphology

SEM (Hitachi S-4800 FEG Scanning Electron Microscope; Hitachi, Tokyo, Japan) was employed to evaluate the morphology of the S. mutans biofilm (n=2 for each group). Each dentin block was fixed in a 2.5% glutaraldehyde solution for 4 h at 4° C. Following dehydration, all the blocks were critical point dried in a desiccator and coated using a sputter coater (Niu et al. 2022).

### 7.5. Effect of GAPI Peptide on Hard Tissue

### 7.5.1. Lesion Depth and Mineral Loss

Micro-CT (SkyScan 1272, Antwerp, Belgium) was employed to evaluate the lesion depths and mineral losses (n=8 for each group). The X-ray source parameters were set at an 80 kV voltage and 100 μA current with an image pixel size of 10 μm. Moreover, two standard mineral cylindrical phantoms (Bruker, Kontich, Belgium) with mineral density values (MDVs, gHApcm⁻³) of 0.25 gHApcm⁻³ and 0.75 gHApcm⁻³ were scanned to calibrate the blocks' greyscales. The NRecon reconstruction software (Version 1.7.4.6, SkyScan, Antwerp, Belgium) was utilized to reconstruct the scanning images. The CTAn data analysis software (CTAn version 1.20.3.0, Skyscan NV, Kontich, Belgium) was employed to analyze the cross-sectional images. The lesion depths were measured, and the images' greyscale values were calibrated. Subsequently, the demineralized and control areas were calculated into the MDV for each block. The mineral loss was determined as the difference between the MDV of the control and the MDV of the demineralized area (Niu et al., 2022).

### 7.5.2. Chemical Structure

FTIR (Spectrum Two, PerkinElmer, Waltham, MA, USA) was employed to examine the changes in the dentin's chemical structures (n=8 for each group). The infrared radiation wavelengths ranged from 550 to 2000 cm⁻¹, the HPO₄²⁻ band wavelength from 900 to 1200 cm⁻¹, and the amide I band wavelength from 1585 to 1720 cm⁻¹. The ratio of the HPO₄²⁻ to amide I absorbance area represented the degree of demineralization in the dentin.

### 7.5.3. Surface Morphology and Cross-Sectional Morphology

SEM (Hitachi S-4800 FEG Scanning Electron Microscope; Hitachi, Tokyo, Japan) was employed to evaluate the dentin surface morphology (n=2 for each group) and cross sectional topography (n=2 for each group). Four blocks from each group were ultrasonically cleaned in distilled water to remove the biofilm prior to its fixation in 2.5% glutaraldehyde at 4° C. for 4 h. Of the four blocks, two were fractured in half in liquid nitrogen for an observation of their cross-sectional topography. A series of ethanol solutions was used to dehydrate the blocks. Subsequently, the blocks were dried in a desiccator and coated with carbon using a sputter coater (Niu et al., 2022).

### 7.5.4. Crystal Characteristics

X-ray diffraction (XRD, Rigaku SmartLab 9 kW with CuKa [1=1.5418 A], Bruker AXS GmbH, Karlsruhe, Germany) was employed to examine the dentin's crystal properties. Scans were conducted with a range of 20-60° 20 and a step size of 0.050 at a scanning speed of 30 s/step. The phase purity and indexing of the chemical phase were verified using the International Centre for Diffraction Data database (PDF-2 Release 2004) (Niu et al. 2022).

### 7.6. Statistical Analysis

All the data were digitalized and analyzed using the SPSS version 23 (IBM Corp, Armonk, NY, USA). The Shapiro-Wilk test was conducted to assess the normality of the distribution. A two-sample t-test was employed to measure the differences in the dead-to-live ratios, log CFUs, lesion depths, mineral losses, and HPO4²⁻ to amide I ratios between the two groups. The significance level was set at 5%.

### 7.7 Results

A total of eighty dentine blocks were prepared for this study. After a 3-day S. mutans biofilm challenge at 37° C., an artificial caries lesion surface, with around a 96 μm depth, was successfully created. Then, all the dentine blocks were allocated into the GAPI-treated group (160 μM GAPI) and the control group (deionized water). All the blocks underwent a biochemical model involving chemical remineralization and a S. mutans biofilm challenge for seven days.

### 7.7.1. Effect of GAPI Peptide on Biofilm

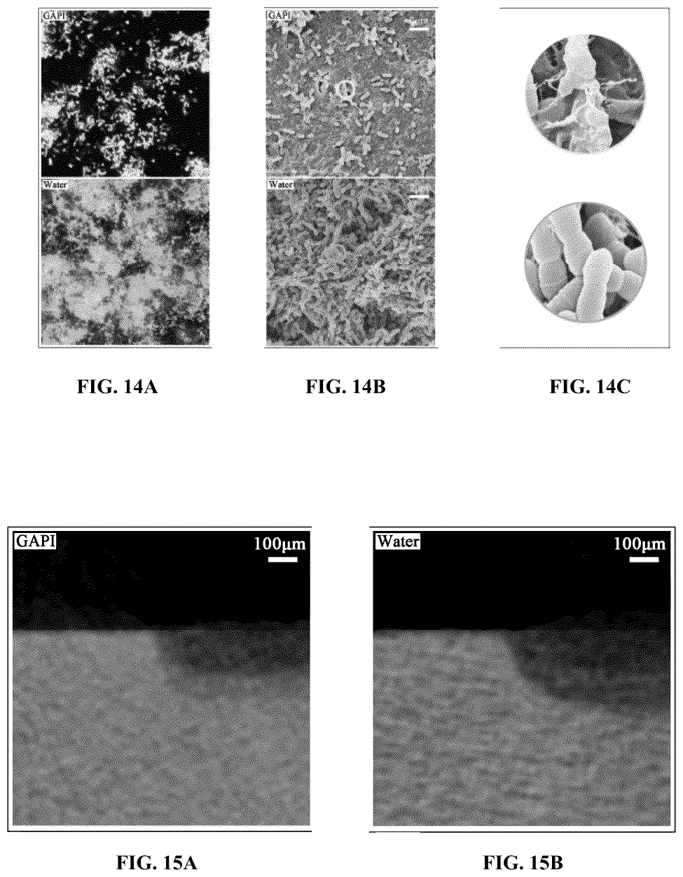

The confocal laser scanning microscopy (CLSM) images displayed an increased red fluorescence of S. mutans on the dentin in the GAPI-treated group. On the other hand, the CLSM images exhibited almost entirely green fluorescence of S. mutans on the dentin in the control group (FIG. 14).

When the dead-live rations from the CLSM images were computed, the ratios for the GAPI and control groups were 0.77±0.13 and 0.37±0.09, respectively (p<0.001). These findings suggested that GAPI significantly hindered the growth of S. mutans on the dentin surface. Table 4 shows the log CFUs representing the growth kinetics of S. mutans in both groups. The log CFUs were 7.45±0.32 and 8.74+0.5(p<0.001) for the GAPI and control groups, respectively.

TABLE 4
S. mutans biofilm and carious dentine
lesions treated with GAPI and water

| Group | GAPI | Water | p Value |
|---|---|---|---|
| S. mutans biofilm assessment | | | |
| Viability: Dead-to-live ratio (n = 8) | 0.77 ± 0.13 | 0.37 ± 0.09 | <0.001 |
| Kinectics: Log₁₀ CFU (n = 8) | 7.45 ± 0.32 | 8.74 ± 0.50 | <0.001 |
| Carious dentine lesion assessment | | | |
| Lesion depth, μm (n = 8) | 151 ± 18 | 214 ± 15 | <0.001 |
| Mineral loss, gHAcm⁻³ (n = 8) | 0.91 ± 0.07 | 1.01 ± 0.07 | 0.01 |
| Hydrogen phosphate-to-amide | 2.92 ± 0.82 | 1.83 ± 0.73 | 0.014 |
| I ratio (n = 8) | | | |

In the low-magnification scanning electron microscopy (SEM) images, the bacterial coverage on the dentin surface in the GAPI group was lower than that in the control group (FIG. 15). In the control group, a three-dimensional S. mutans biofilm entirely covered the dentin surface, with the bacterial cells being tightly connected to each other. In the high magnification SEM images, the GAPI treatment disrupted the bacterial structure, including a loss of normal bacterial cell morphology and damage to the bacterial cell membranes. Conversely, the bacterial cells remained intact in the control group.

### 7.7.2 Effects of GAPI Peptide on Hard Tissue

Micro-computed tomography (micro-CT), Fourier transform infrared (FTIR), SEM, and X-ray diffraction (XRD) were used to assess the lesion depths, mineral losses, changes in chemical structure, dentine surfaces and cross-section morphologies, and crystal characteristics on the dentine blocks' carious lesions, respectively. Table 4 presents the average lesion depth and average mineral loss. The lesion depths for the GAPI and control groups were 151±18 m and 214±15 m, respectively (p<0.001). The mineral losses for the GAPI and control groups were 0.91±0.07 gHAcm-3 and 1.01±0.07 gHAcm⁻³, respectively (p=0.01). FIG. 15 shows the representative micro-CT images. The dentin blocks treated with GAPI exhibited a notably lower average lesion depth and mineral loss compared to those of the control group.

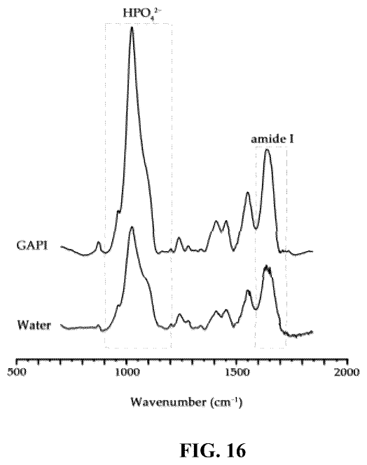

The average HPO₄²⁻-to-amide I ratios for the GAPI and control groups were 2.92±0.82 and 1.83±0.73 (p=0.014). This indicates that the dentin in the control group experienced a greater mineral tissue loss than that in the GAPI group. FIG. 16 displays the characteristic Fourier transform infrared spectroscopy (FTIR) spectra of both groups.

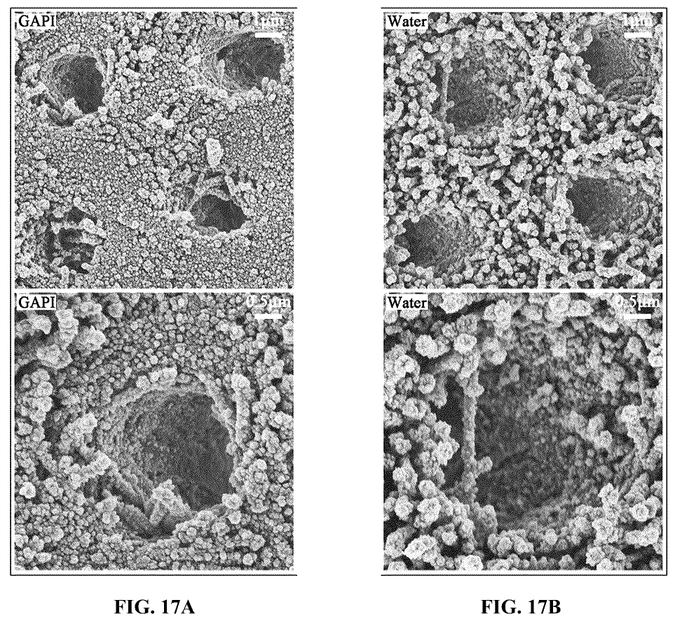

FIG. 17 displays the typical dentin surface morphology. In the GAPI group, the dentin surface was relatively smooth, with slight collagen fiber exposure. In addition, the ballform mineral nodes on the dentine in the GAPI group indicated remineralization. In contrast, the dentin surface appeared to be rough, and the exposure of collagen fibers was significantly evident in the control group.

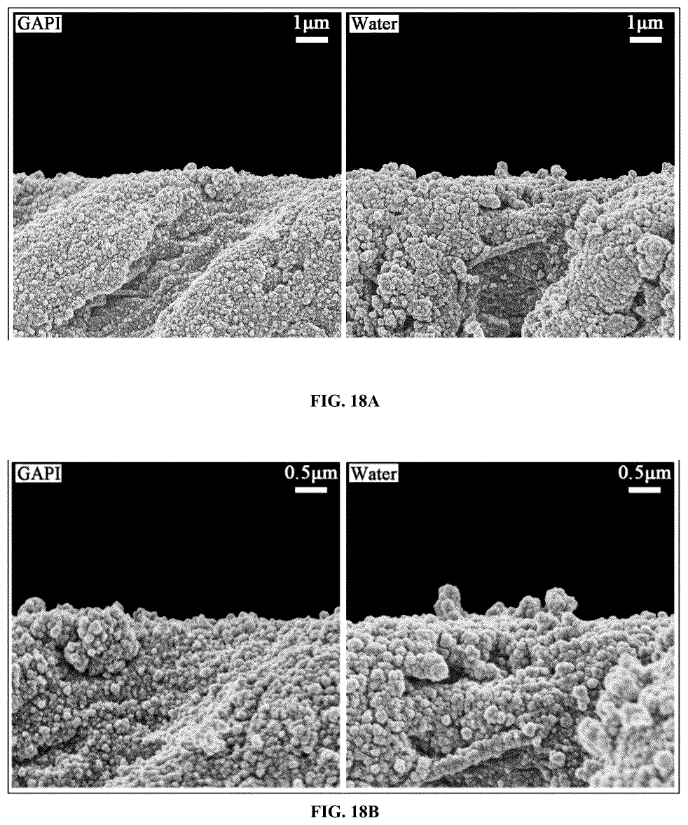

FIG. 18 shows typical dentine's cross-sectional morphology. The cross-sectional images exhibited that the dentinal tubular and inter-tubular regions were relatively full of mineral nodes, with slight dentin collagen fiber exposure in the GAPI group

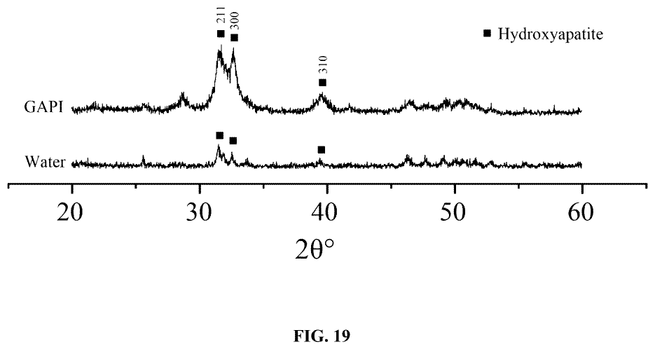

FIG. 19 displays the typical characteristic XRD spectra of dentin. In the GAPI group, hydroxyapatite diffraction peaks (211, 300, and 310) were identified at 31.8, 32.9, and 39.8. These peaks suggested the existence of hydroxyapatite crystals. In contrast, the spectra in the control group showed no prominent diffraction peaks.

### 7.8 Discussion

Recently, antimicrobial peptides have been regarded as a bioactive material for developing novel anti-caries agents (Raheem et al., 2019). Polyphemusin I is a broad-spectrum antimicrobial peptide used against S. mutans (Zhang et al. 2019). It could be an ideal template peptide for developing novel anti-caries peptides. Gallic acid with a pyrogallol group can attract calcium from the microenvironment to enhance hydroxyapatite regeneration. In a previous study, we developed and synthesized a novel peptide, GAPI, by attaching gallic acid to polyphemusin I. It is essential to investigate GAPI's impact on dentine caries, considering its antibiofilm and remineralizing effects. We showed that GAPI possesses antimicrobial and mineralizing properties and is capable of inhibiting the growth of cariogenic biofilms and promoting the remineralization of initial enamel caries in a chemical model. However, chemical models, such as pH-cycling models, lack biological factors. Thus, we utilized a biofilm remineralization cycling model involving S. mutans in this study. This unique model incorporated both biological and chemical factors to create pH changes and establish a microbiological environment that mimicked bacterial impacts in the oral cavity. The effectiveness of this model in simulating the oral environment was demonstrated. The S. mutans biofilm is a frequently employed microbial model for caries research. In this study, we utilized an S. mutans monospecies biofilm challenge to induce demineralization and create initial dentine caries, which was then incorporated into the S. mutans biofilm remineralization cycling model. In order to produce acid, 5% sucrose was added to the bacterial culture. Furthermore, to mimic a high-risk caries scenario, we subjected the dentine to a 16 h demineralization process. GAPI's antibiofilm effect on the S. mutans biofilm on the dentin surface was evaluated by assessing the viability, growth kinetics, and morphology. A quantitative analysis of the dead-live ratio and log CFUs revealed a significant difference between the two groups. These findings suggested that GAPI could effectively inhibit the growth of the S. mutans biofilm on the dentin. The SEM images of the biofilm further confirmed that GAPI could damage the bacterial cell membrane, resulting in the loss of a typical bacterial structure and the leakage of cytoplasmic content. This mechanism was related to the antimicrobial function domain, polyphemusin I, which is derived from horseshoe crabs with a positive net charge [24]. Amiss et al. indicated that polyphemusin I can effectively kill bacteria with lower concentrations in extracellular and intracellular environments (Amiss et al., 2021). Polyphemusin I is known as one of the cell-penetrating peptides due to its—hairpin structure. Cell penetrating peptides typically consist of 5-30 amino acids, allowing them to cross cell membranes and interact with intracellular targets (Guidotti et al., 2017). The results suggest that the GAPI peptide has potential as an antimicrobial agent for caries management. GAPI's mineralizing effects on dentin caries manifested in promoting remineralization, impeding demineralization, and preventing collagen degradation. These effects could be examined through lesion depth, mineral loss, chemical structure, crystal properties, and surface morphology. Cross-sectional morphology can help to explain collagen degradation.

In this study, micro-CT, a non-invasive testing technique, was used to assess the dentin's mineral content. The micro-CT results, such as those for lesion depth and mineral loss, demonstrated less demineralization in the GAPI group. Moreover, we used FTIR to analyze the dentin blocks' chemical structures. In the FTIR spectra, the amide I band represented collagen and the phosphate band signified the mineral matrix. The HPO₄²⁻-to-amide I ratio was correlated with the degree of demineralization, with higher ratios indicating reduced demineralization. In this study, the HPO₄²⁻-to-amide I ratio in the GAPI group was significantly higher than that in the control group. In addition, the amide I band area in the GAPI group exceeded than that in the control group. These findings indicated that GAPI effectively inhibited demineralization and collagen degradation. The observed effect of impeding demineralization and preventing collagen degradation can be partly ascribed to the lower bacterial burden given by the antimicrobial activity of the peptide. Moreover, the SEM results revealed the presence of mineral nodes in the GAPI group, confirming that GAPI promoted the formation of extra-fibrillar minerals. Due to dentin's unique composition, the collagen structure plays a crucial role as a scaffold for the deposition of mineral crystals. Mineral crystals and calcium resources are also essential. Therefore, these factors need to be considered in dentin caries treatment. Fluoride-based materials, such as silver diamine fluoride, are commonly applied in caries management and can be incorporated into fluorapatite crystals, enhancing the remineralizing process [8]. However, this may lead to black discoloration on the tooth (Zhang et al., 2023). Other bioactive substances, such as calcium- and phosphate-based materials, including casein phosphopeptide-amorphous calcium phosphate and nano-hydroxyapatite, act as sources of calcium and phosphate for remineralizing the demineralized dentin surface (Gonzalez-Cabezas et al., 2018). GAPI can suppress the growth of cariogenic biofilms, thereby reducing acid production. This mechanism can hinder the dissolution of hydroxyapatite and prevent collagen degradation. In addition, the pyrogallol group of GAPI can help to attract calcium and phosphate to promote remineralization. In conclusion, the peptide GAPI is capable of hindering S. mutans biofilm development on dentine surfaces. In addition, GAPI promoted remineralization, impeded demineralization, and prevented the collagen degradation of the dentine.

### Exemplary Products, Systems and Methods are Set Out in the Following Items:

1. A conjugate of polyphemusin-1 and gallic acid (GAPI).
2. The conjugate of item 1, wherein the polyphmusin-1 is conjugated to the gallic acid directly or through a linker.
3. The conjugate of any one of the preceding items, wherein the gallic acid is conjugated to the amino terminus of polyphemusin-1.
4. The conjugate of any one of the preceding items, represented by the structure (SEQ ID NO: 11):

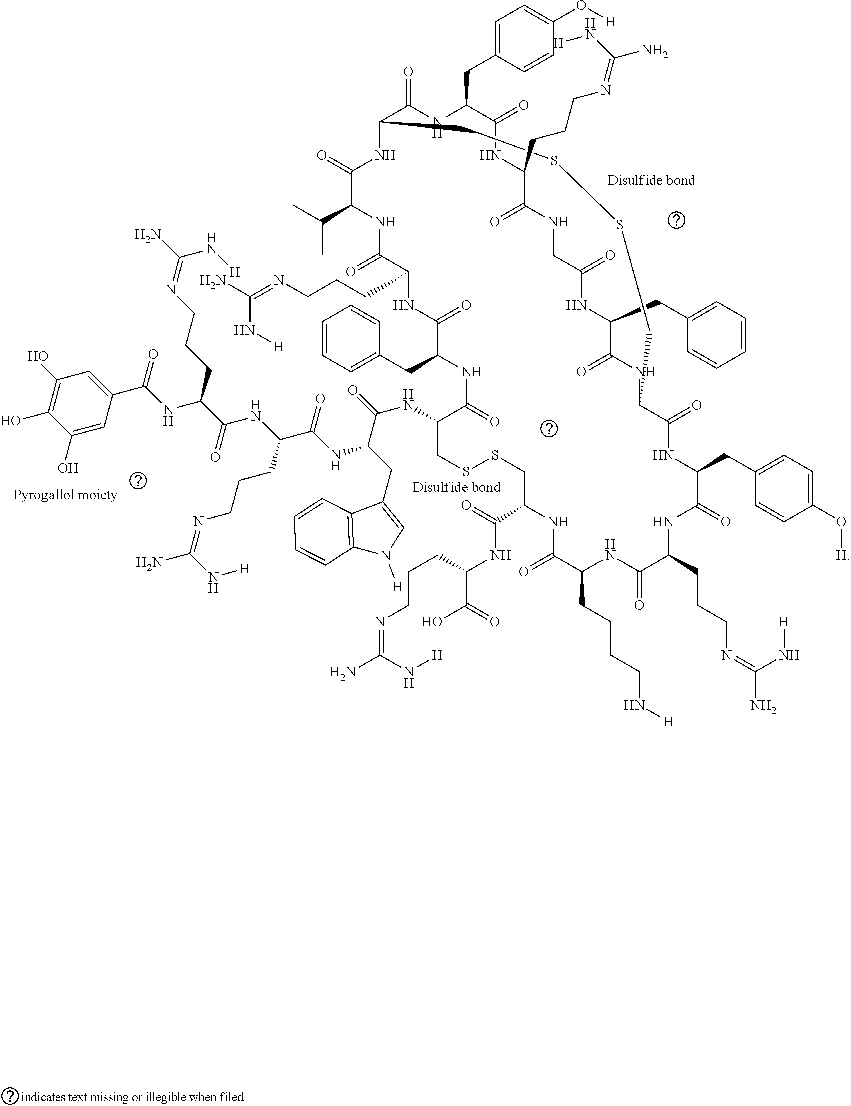

5. The conjugate of any one of the preceding items having a purity percentage from 90.0% to 99.9%.
6. The conjugate of any one of the preceding items, wherein the conjugate is at least 90% pure.
7. The conjugate of any one of the preceding items, wherein the conjugate is at least in part in a form of a b-sheet.
8. The conjugate of any one of the preceding items, wherein the conjugate inhibits cariogenic bacteria.
9. The conjugate of any one of the preceding items, wherein the conjugate prevents, inhibits or reduces progression of tooth demineralization, or wherein the conjugate enhances or increases tooth remineralization.
10. The conjugate of any one of the preceding items having a molecular weight ranges from about 2600 kD to 2624 kD with a proportion of the β-sheet of from 40.0% to 56.0%.
11. The conjugate of any one of the preceding items wherein the molecule comprises antimicrobial and remineralizing properties.
12. The conjugate of any one of the preceding items that is stable for at least 60 minutes in saliva and maintain more than 90% of the conjugate in saliva after 60 minutes.
13. A method of making Gallic-Acid-polyphemusin-1 (“GAPI”) molecule comprising reacting gallic acid with Polyphemusin-I and forming disulfide bonds at C4-C17 and C8-C13.
14. The method of item 13 wherein the GAPI has a purity percentage of 90.0% to 99.9%.
15. The method of any one of the preceding items wherein the GAPI has a molecular weight ranges from about 2600 kD to 2624 kD with a proportion of the β-sheet of about 40.0% to 56.0%.
16. The method of any one of the preceding items wherein the conjugate comprises antimicrobial and remineralizing properties.
17. The method of any one of the preceding items wherein the conjugate is stable for at least 60 minutes in saliva and maintain more than 90% in saliva after 60 minutes.
18. A method of inhibiting cariogenic biofilm and remineralizing dentine in a subject, said method comprises administration of an effective amount of the molecule of any one of the preceding items.
19. The method of any one of the preceding items wherein the remineralizes dentine occurs in early enamel caries.
20. The method of any one of the preceding items wherein the method further prevents collagen degradation of dentine.
21. The method of any one of the preceding items wherein the cariogenic biofilm is produced by cariogenic species selected from the group consisting of Streptococcus mutans, Lacticaseibacillus casei and Candida albicans.
22. The method of any one of the preceding items wherein the remineralizing dentine is measured by a chemical pH cycling model comprising Micro-CT scans.
23. The method of any one of the preceding items wherein minimum inhibitory concentration (“MIC”) against Streptococcus mutans (ATCC 35668), Streptococcus mutans (UA159), Lacticaseibacillus casei and Candida albicans is 80 μM, 40 μM, 40 μM and 20 μM, respectively and minimum bactericidal concentration (“MBC”) is 160 μM, 80 μM, 160 μM and 40 μM, respectively.
24. The method of any one of the preceding items wherein MICs against S. mutans and S. sobrinus are 80 μM, wherein the MBCs are 160 μM and 320 μM, respectively.
25. The method of any one of the preceding items wherein MICs against L. acidophilus and L. rhamnosus are 40 μM and 20 μM, and the MBCs are 80 μM and 160 μM, respectively.
26. The method of any one of the preceding items wherein MICs and MBCs for A. naeslundii and E. faecalis are 160 μM and 640 μM, respectively.
27. The method of any one of the preceding items wherein MICs for P. gingivalis and A. actinomycetemcomitans are 320 μM and 160 μM, respectively and the MBCs for P. gingivalis and A. actinomycetemcomitans are 640 μM and 320 μM, respectively.
28. The method of any one of the preceding items wherein MICs and MBCs against cariogenic species range from 20 to 320 μM and 80 to 640 μM, respectively.
29. The method of any one of the preceding items wherein the molecule is stable for at least 60 min in saliva and maintain more than 90% in saliva after 60 minutes.
30. The method of any one of the preceding items wherein the cariogenic biofilm lost its three-dimensional structure upon administration of the molecule.
31. A method of preventing, treating or reducing the incidence of dental caries in a subject in need thereof, comprising administering to the subject the conjugate according to any one of items 1 to 12.
32. The method of item 31, wherein the dental caries is caused by cariogenic bacteria.
33. The method of any one of item 31 or 32, wherein the cariogenic bacteria is selected from the group consisting of Streptococcus, Lactobacillus and Actinomyces.
34. The method of any one of items 31-33, wherein the cariogenic bacteria is selected from the group consisting of Streptococcus mutans, Lacticaseibacillus casei and Candida albicans.
35. The method of any one of items 31-34, wherein the minimum inhibitory concentration (“MIC”) against Streptococcus mutans (ATCC 35668), Streptococcus mutans (UA159), Lacticaseibacillus casei and Candida albicans is 80 μM, 40 μM, 40 μM and 20 μM, respectively.
36. The method of any one of items 31-35, wherein the minimum bactericidal concentration (“MBC”) against Streptococcus mutans (ATCC 35668), Streptococcus mutans (UA159), Lacticaseibacillus casei and Candida albicans is 160 μM, 80 μM, 160 μM and 40 μM, respectively.
37. The method of any one of items 31-36, wherein the dental caries demineralizes or degrades enamel or dentin in a tooth of the subject.
38. The method of any one of items 31-37, wherein the conjugate prevents, inhibits or reduces progression of tooth demineralization in the subject.
39. The method of any one of items 31-38, wherein the conjugate enhances or increases tooth remineralization in the subject.
40. The method of any one of items 31-39, wherein the conjugate inhibits cariogenic bacteria, or decreases adherence of cariogenic bacteria to tooth enamel or dentin in the subject.
41. The method of any one of items 31-40, wherein the conjugate is administered in a pharmaceutical composition suitable for dental administration.
42. A method of inhibiting cariogenic biofilm in a subject in need thereof, said method comprising administering to the subject an effective amount of the conjugate of any one of items 1-12.
43. A method of preventing, inhibiting or reducing the progression of enamel or dentin demineralization or degradation in a subject in need thereof, comprising administering to the subject an effective amount of the conjugate of any one of items 1-12.
44. A method of remineralizing dentin or enamel in a subject in need thereof, comprising administering to the subject an effective amount of the compound according to any one of items 1-12.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments but should be defined only in accordance with the following claims and their equivalents.

All references cited herein are incorporated herein by reference in their entirety and for all purposes to the same extent as if each individual publication or patent or patent application was specifically and individually indicated to be incorporated by reference in its entirety for all purposes.

### REFERENCES

Alvarez G, Gonzalez M, Isabal S, Blanc V, Leon R. 2013. Method to quantify live and dead cells in multi-species oral biofilm by real-time per with propidium monoazide. AMB Express. 3(1):1.
Amiss, A. S.; von Pein, J. B.; Webb, J. R.; Condon, N. D.; Harvey, P. J.; Phan, M.-D.; Schembri, M. A.; Currie, B. J.; Sweet, M. J.; Craik, D. J.; et al. Modified horseshoe crab peptides target and kill bacteria inside host cells. Cell. Mol. Life Sci. 2021, 79, 38.
Andersson D I, Hughes D, Kubicek-Sutherland J Z. 2016. Mechanisms and consequences of bacterial resistance to antimicrobial peptides. Drug Resist Updat. 26:43-57.
Cai M, Sugumaran M, Robinson W E. 2008. The crosslinking and antimicrobial properties of tunichrome. Comp Biochem Physiol B Biochem Mol Biol. 151(1):110-117.
Cao Y, Liu W, Ning T, Mei M L, Li Q L, Lo E C, Chu C H. 2014. A novel oligopeptide simulating dentine matrix protein 1 for biomimetic mineralization of dentine. Clin Oral Investig. 18(3):873-881.
Caufield P W, Schon C N, Saraithong P, Li Y, Argimon S. 2015. Oral lactobacilli and dental caries: A model for niche adaptation in humans. J Dent Res. 94(9 Suppl):110S-1185.
Eckert R, He J, Yarbrough D K, Qi F, Anderson M H, Shi W. 2006. Targeted killing of Streptococcus mutans by a pheromone-guided “smart” antimicrobial peptide. Antimicrob Agents Chemother. 50(11):3651-3657.
Chen, X.; Daliri, E. B.; Kim, N.; Kim, J. R.; Yoo, D.; Oh, D. H. Microbial Etiology and Prevention of Dental Caries: Exploiting Natural Products to Inhibit Cariogenic Biofilms. Pathogens 2020, 9, 569.
Edwards I A, Elliott A G, Kavanagh A M, Zuegg J, Blaskovich M A, Cooper M A. 2016.
Contribution of amphipathicity and hydrophobicity to the antimicrobial activity and cytotoxicity of beta-hairpin peptides. ACS Infect Dis. 2(6):442-450.
González-Cabezas, C.; Fernández, C. Recent Advances in Remineralization Therapies for Caries Lesions. Adv. Dent. Res. 2018, 29, 55-59.
Guidotti, G.; Brambilla, L.; Rossi, D. Cell-Penetrating Peptides: From Basic Research to Clinics. Trends Pharmacol. Sci. 2017, 38, 406-424.
Huang Z B, Shi X, Mao J, Gong S Q. 2016. Design of a hydroxyapatite-binding antimicrobial peptide with improved retention and antibacterial efficacy for oral pathogen control. Sci Rep. 6:38410.
Kassebaum, N.J.; Smith, A. G. C.; Bernabe, E.; Fleming, T. D.; Reynolds, A. E.; Vos, T.; Murray, C. J. L.; Marcenes, W.; GBD 2015 Oral Health Collaborators. Global, Regional, and National Prevalence, Incidence, and Disability-Adjusted Life Years for Oral Conditions for 195 Countries, 1990-2015: A Systematic Analysis for the Global Burden of Diseases, Injuries, and Risk Factors. J. Dent. Res. 2017, 96, 380-387.
Koo H, Bowen W H. 2014. Candida albicans and Streptococcus mutans: A potential synergistic alliance to cause virulent tooth decay in children. Future Microbiol. 9(12):1295-1297.
Krzysciak W, Koscielniak D, Papiez M, Vyhouskaya P, Zagorska-Swiezy K, Kolodziej I, Bystrowska B, Jurczak A. 2017. Effect of a Lactobacillus salivarius probiotic on a double-species Streptococcus mutans and Candida albicans caries biofilm. Nutrients. 9(11).
Listl, S.; Galloway, J.; Mossey, P. A.; Marcenes, W. Global Economic Impact of Dental Diseases. J. Dent. Res. 2015, 94, 1355-1361.
Loozen G, Boon N, Pauwels M, Quirynen M, Teughels W. 2011. Live/dead real-time polymerase chain reaction to assess new therapies against dental plaque-related pathologies. Mol Oral Microbiol. 26(4):253-261.
Luo Y, Song Y. 2021. Mechanism of antimicrobial peptides: Antimicrobial, anti-inflammatory and antibiofilm activities. Int J Mol Sci. 22(21).
Lv Y, Wang J, Gao H, Wang Z, Dong N, Ma Q, Shan A. 2014. Antimicrobial properties and membrane-active mechanism of a potential alpha-helical antimicrobial derived from cathelicidin pmap-36. PLoS One. 9(1):e86364.
Mai S, Mauger M T, Niu L N, Barnes J B, Kao S, Bergeron B E, Ling J Q, Tay F R. 2017. Potential applications of antimicrobial peptides and their mimics in combating caries and pulpal infections. Acta Biomater. 49:16-35.
Mei M L, Li Q L, Chu C H, Lo E C, Samaranayake L P. 2013. Antibacterial effects of silver diamine fluoride on multi-species cariogenic biofilm on caries. Ann Clin Microbiol Antimicrob. 12:4.
Niu J Y, Yin I X, Mei M L, Wu W K K, Li Q L, Chu C H. 2021a. The multifaceted roles of antimicrobial peptides in oral diseases. Mol Oral Microbiol. 36(3):159-171.
Niu J Y, Yin I X, Wu W K K, Li Q L, Mei M L, Chu C H. 2021b. Antimicrobial peptides for the prevention and treatment of dental caries: A concise review. Arch Oral Biol. 122:105022.
Niu J Y, Yin I X, Wu W K K, Li Q L, Mei M L, Chu C H. 2021c. Data from: A concise review on antimicrobial peptides for prevention and treatment of dental caries. In: Dryad, editor. Dryad.
Niu J Y, Yin I X, Wu W K K, Li Q L, Mei M L, Chu C H. 2021d. A novel dual-action antimicrobial peptide for caries management. J Dent. 111:103729.
Niu J Y, Yin I X, Wu W K K, Li Q L, Mei M L, Chu C H. 2022. Efficacy of the dual-action ga-krl2 peptide for remineralising initial enamel caries: An in vitro study. Clin Oral Investig. 26(3):2441-2451.
Peres, M. A.; Macpherson, L. M. D.; Weyant, R. J.; Daly, B.; Venturelli, R.; Mathur, M. R.; Listl, S.; Celeste, R. K.; Guarnizo-Herreno, C. C.; Kearns, C.; et al. Oral diseases: A global public health challenge. Lancet 2019, 394, 249-260.
Pitts N B, Zero D T, Marsh P D, Ekstrand K, Weintraub J A, Ramos-Gomez F, Tagami J, Twetman S, Tsakos G, Ismail A. 2017. Dental caries. Nat Rev Dis Primers. 3:17030.
Powers J P, Martin M M, Goosney D L, Hancock R E. 2006. The antimicrobial peptide polyphemusin localizes to the cytoplasm of Escherichia coli following treatment. Antimicrob Agents Chemother. 50(4):1522-1524.
Powers J P, Rozek A, Hancock R E. 2004. Structure-activity relationships for the beta-hairpin cationic antimicrobial peptide polyphemusin i. Biochim Biophys Acta. 1698(2):239-250.
Prajatelistia E, Ju S W, Sanandiya N D, Jun S H, Ahn J S, Hwang D S. 2016. Tunicate-inspired gallic acid/metal ion complex for instant and efficient treatment of dentin hypersensitivity. Adv Healthc Mater. 5(8):919-927.
Raheem, N.; Straus, S. K. Mechanisms of Action for Antimicrobial Peptides with Antibacterial and Antibiofilm Functions. Front. Microbiol. 2019, 10, 2866.
Reis A C M, Bezerra D D S, Hart-Chu E N S, Stipp R N, Guedes S F F, Neves B G, Rodrigues L K A. 2021. Quantification and gene expression of Lactobacillus casei group species associated with dentinal lesions in early childhood caries. Saudi Dent J. 33(2):69-77.
Sullivan R, Santarpia P, Lavender S, Gittins E, Liu Z, Anderson M H, He J, Shi W, Eckert R. 2011. Clinical efficacy of a specifically targeted antimicrobial peptide mouth rinse: Targeted elimination of Streptococcus mutans and prevention of demineralization. Caries Res. 45(5):415-428.
Takahashi Y, Yoshida A, Nagayoshi M, Kitamura C, Nishihara T, Awano S, Ansai T. 2011. Enumeration of viable Enterococcus faecalis, a predominant apical periodontitis pathogen, using propidium monoazide and quantitative real-time polymerase chain reaction. Microbiol Immunol. 55(12):889-892.
Taniguchi M, Ochiai A, Matsushima K, Tajima K, Kato T, Saitoh E, Tanaka T. 2016. Endotoxin-neutralizing activity and mechanism of action of a cationic alpha-helical antimicrobial octadecapeptide derived from alpha-amylase of rice. Peptides. 75:101-108.
Tao R, Jurevic R J, Coulton K K, Tsutsui M T, Roberts M C, Kimball J R, Wells N, Berndt J, Dale B A. 2005. Salivary antimicrobial peptide expression and dental caries experience in children. Antimicrob Agents Chemother. 49(9):3883-3888.
Tonetti, M. S.; Bottenberg, P.; Conrads, G.; Eickholz, P.; Heasman, P.; Huysmans, M. C.; Lopez, R.; Madianos, P.; Muller, F.; Needleman, I.; et al. Dental caries and periodontal diseases in the ageing population: Call to action to protect and enhance oral health and well-being as an essential component of healthy ageing-Consensus report of group 4 of the joint EFP/ORCA workshop on the boundaries between caries and periodontal diseases. J. Clin. Periodontol. 2017, 44 (Suppl. 18), S135-S144.
Wang C, van der Mei H C, Busscher H J, Ren Y. 2020. Streptococcus mutans adhesion force sensing in multi-species oral biofilms. NPJ Biofilms Microbiomes. 6(1):25.
Wiegand I, Hilpert K, Hancock R E. 2008. Agar and broth dilution methods to determine the minimal inhibitory concentration (mic) of antimicrobial substances. Nat Protoc. 3(2):163-175.
Yassin S A, German M J, Rolland S L, Rickard A H, Jakubovics N S. 2016. Inhibition of multispecies biofilms by a fluoride-releasing dental prosthesis copolymer. J Dent. 48:62-70.
Yeaman M R, Yount N Y. 2003. Mechanisms of antimicrobial peptide action and resistance. Pharmacol Rev. 55(1):27-55.
Yu O Y, Mei M L, Zhao I S, Li Q L, Lo E C, Chu C H. 2018. Remineralisation of enamel with silver diamine fluoride and sodium fluoride. Dent Mater. 34(12):e344-e352.
Zhang L Y, Fang Z H, Li Q L, Cao C Y. 2019. A tooth-binding antimicrobial peptide to prevent the formation of dental biofilm. J Mater Sci Mater Med. 30(4):45.
Zhang M, Wei W, Sun Y, Jiang X, Ying X, Tao R, Ni L. 2016. Pleurocidin congeners demonstrate activity against streptococcus and low toxicity on gingival fibroblasts. Arch Oral Biol. 70:79-87.
Zhang O L, Niu J Y, Yin I X, Yu O Y, Mei M L, Chu C H. 2022. Growing global research interest in antimicrobial peptides for caries management: A bibliometric analysis. Journal of Functional Biomaterials. 13(4).
Zhang O L, Niu J Y, Yin I X, Yu O Y, Mei M L, Chu C H. 2023a. Bioactive materials for caries management: A literature review. Dent J (Basel). 11(3).
Zhang O L, Niu J Y, Yu O Y, Mei M L, Jakubovics N S, Chu C H. 2023b. Peptide designs for use in caries management: A systematic review. Int J Mol Sci. 24(4).
Zhang, L. Y.; Fang, Z. H.; Li, Q. L.; Cao, C. Y. A tooth-binding antimicrobial peptide to prevent the formation of dental biofilm. J. Mater. Sci. Mater. Med. 2019, 30, 45.
Zhou Y Z, Cao Y, Liu W, Chu C H, Li Q L. 2012. Polydopamine-induced tooth remineralization. ACS Appl Mater Interfaces. 4(12):6901-6910.

1. A conjugate of polyphemusin-1 and gallic acid (GAPI).

2. The conjugate of claim 1, wherein the polyphemusin-1 is conjugated to the gallic acid directly or through a linker.

3. The conjugate of claim 1, wherein the gallic acid is conjugated to the amino terminus of polyphemusin-1.

4. The conjugate of claim 1, represented by the structure (SEQ ID NO: 11): 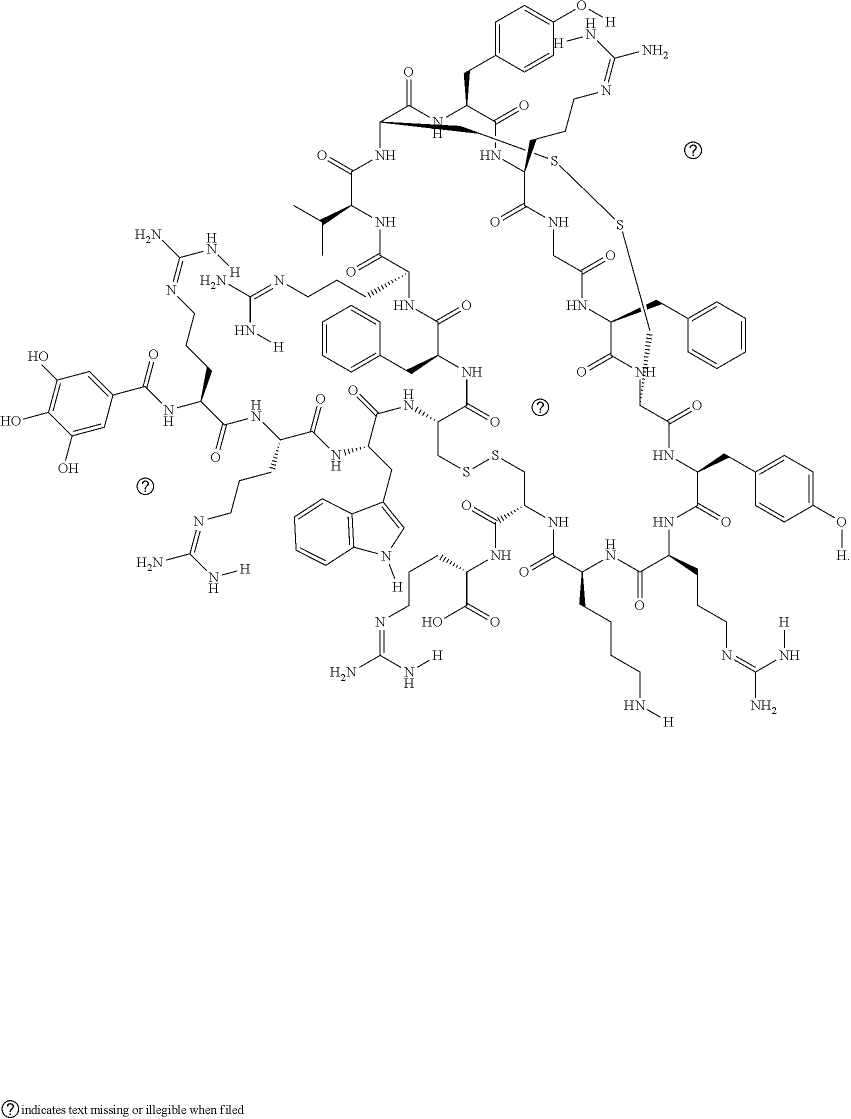
or a salt thereof.

5. The conjugate of claim 1, wherein the conjugate is at least 90% pure.

6. The conjugate of claim 1, wherein the conjugate is at least in part in a form of a β-sheet.

7. The conjugate of claim 1, wherein the conjugate inhibits cariogenic bacteria.

8. The conjugate of claim 1, wherein the conjugate prevents, inhibits or reduces progression of tooth demineralization, or wherein the conjugate enhances or increases tooth remineralization.

9. The conjugate of claim 1, wherein the conjugate is stable for at least 60 minutes in saliva, and maintains more than 90% of the conjugate in saliva after 60 minutes.

10. A method of preventing, treating or reducing the incidence of dental caries in a subject in need thereof, comprising administering to the subject the conjugate according to claim 1.

11. The method of claim 10, wherein the dental caries is caused by cariogenic bacteria.

12. The method of claim 11, wherein the cariogenic bacteria is selected from the group consisting of Streptococcus, Lactobacillus and Actinomyces.

13. The method of claim 12, wherein the cariogenic bacteria is selected from the group consisting of Streptococcus mutans, Lacticaseibacillus casei and Candida albicans.

14. The method of claim 13, wherein the minimum inhibitory concentration (“MIC”) against Streptococcus mutans (ATCC 35668), Streptococcus mutans (UA159), Lacticaseibacillus casei and Candida albicans is 80 μM, 40 μM, 40 μM and 20 μM, respectively.

15. The method of claim 13, wherein the minimum bactericidal concentration (“MBC”) against Streptococcus mutans (ATCC 35668), Streptococcus mutans (UA159), Lacticaseibacillus casei and Candida albicans is 160 μM, 80 μM, 160 μM and 40 μM, respectively.

16. The method of claim 10, wherein the dental caries demineralizes or degrades enamel or dentin in a tooth of the subject.

17. The method of claim 10, wherein the conjugate prevents, inhibits or reduces progression of tooth demineralization in the subject.

18. The method of claim 10, wherein the conjugate enhances or increases tooth remineralization in the subject.

19. The method of claim 10, wherein the conjugate inhibits cariogenic bacteria, or decreases adherence of cariogenic bacteria to tooth enamel or dentin in the subject.

20. The method of claim 10, wherein the conjugate is administered in a pharmaceutical composition suitable for dental administration.

21. A method of inhibiting cariogenic biofilm in a subject in need thereof, said method comprising administering to the subject an effective amount of the conjugate of claim 1.

22. A method of preventing, inhibiting or reducing the progression of enamel or dentin demineralization or degradation in a subject in need thereof, comprising administering to the subject an effective amount of the conjugate of claim 1.

23. A method of remineralizing dentin or enamel in a subject in need thereof, comprising administering to the subject an effective amount of the compound according to claim 1.
